SEQUENCE LISTING (6 sequences)

SEQ ID NO: 1 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 2 - protein, 220 aa, Homo sapiens
ZVQLEQSGPGLVRPSQTLSLTCTVSGTSFDDYYWTWVRQPPGRGLEWIGYVFYTGTTLLDPSLRGRVTMLVNTSKNQFSLRLSSVTAADTAVYYCARNLIAGGIDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - DNA, 684 bp, Homo sapiens
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaatga

SEQ ID NO: 4 - DNA, 689 bp, Homo sapiens
gtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggctagctgg

SEQ ID NO: 5 - DNA, 684 bp, Homo sapiens
gacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatga

SEQ ID NO: 6 - DNA, 674 bp, Homo sapiens
cccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatg

(19) United States
(12) Patent Application Publication
(10) Pub. No.: US 2018/0021455 A1
(43) Pub. Date: Jan. 25, 2018

(54) RECEPTOR-TARGETED NANOPARTICLES FOR ENHANCED TRANSCYTOSIS MEDIATED DRUG DELIVERY

(71) Applicants: The Brigham and Women's Hospital, Inc., Boston, MA (US); Massachusetts Institute of Technology, Cambridge, MA (US)

(72) Inventors: Eric M. Pridgen, Stanford, CA (US); Frank Alexis, Greenville, SC (US); Omid C. Farokhzad, Waban, MA (US); Robert S. Langer, Newton, MA (US); Richard S. Blumberg, Waltham, MA (US)

(21) Appl. No.: 15/692,116

(22) Filed: Aug. 31, 2017

Related U.S. Application Data
(63) Continuation of application No. 15/039,770, filed on May 26, 2016.
Application No. 15/039,770 is a continuation-in-part of application No. 12/515,465, filed on May 5, 2010.
(60) Provisional application No. 61/909,261, filed on Nov. 26, 2013.
(60) Provisional application No. 61/909,663, filed on Nov. 27, 2013.
(60) Provisional application No. 60/860,043, filed on Nov. 20, 2006.

(51) Int. Cl. A61K 9/50; A61K 9/00; C07K 16/28; A61K 38/28; A61K 39/00

(52) U.S. Cl. CPC A61K 47/6931; A61K 9/5089; A61K 47/68; A61K 47/6935; A61K 47/6849; C07K 16/283; A61K 38/28; A61K 47/6929; A61K 9/50; A61K 9/0053; A61K 2039/505; C07K 2317/52; C07K 2317/524; C07K 2317/526

(57) ABSTRACT

Receptor-targeted nanoparticles (R-NPs) are provided for selective transport into and through targeted tissues of therapeutic, prophylactic and diagnostic agents. R-NPs can include polymeric particle, lipid particles, inorganic particles, or a combination thereof with a targeting moiety selective for binding to a receptor on the cells where the agent is to be delivered, where the receptor mediates transcytosis of the nanoparticle into and through the cells. In a preferred embodiment, the targeting moiety is the neonatal Fc receptor. Examples demonstrate Fc-targeted nanoparticles which are actively transported across the intestinal epithelium, providing a route for the oral delivery of nanoparticle encapsulated active agents including peptides such as insulin.

### STATEMENT REGARDING FEDERALLY SPONSORED RESEARCH OR DEVELOPMENT

This invention was made with government support under Grant No. EB015419-01 awarded by the National Institute of Health (NIH) and Grant No. DK53056 awarded by the NIH. The government has certain rights in the invention.

### FIELD OF THE INVENTION

This invention is generally in the field of compositions for receptor mediated targeted drug delivery through tissue, such as enhanced delivery to the gastrointestinal tract of agents that are difficult to administer orally especially peptides such as insulin and through cellular barriers such as the blood brain barrier.

### BACKGROUND OF THE INVENTION

There are many drugs that would be safer and more efficacious if they could be selectively administered systemically. For example, many chemotherapeutics are extremely toxic to both cancer cells and normal cells. In most cases, there is no way to bias systemic delivery to the cancer cells, particularly if specific tumor receptors are not known and available.

Oral administration of therapeutics is the standard of care to increase patient compliance, decrease cost of administration and provide sustained, delayed or pulsatile administration. Patients prefer the convenience of oral administration relative to parenteral administration. Nano or microparticulate formulations are not readily absorbed when administered orally and are therefore administered primarily by injection. For NP-based therapeutics to be a practical treatment for many diseases, NP formulations appropriate for oral administration are necessary. The most significant barrier to the effective oral administration of NPs is the intestinal epithelium, which limits the absorption of NPs. To date, there is no practical solution to this problem.

There have been many attempts to develop oral drug delivery systems that overcome this barrier (Chen et al., Biomaterials 32:9826-9838 (2011)). For example, permeation enhancers have been used to open tight junctions to allow both paracellular and transcellular transport of drugs across the epithelium (Salama et al., Adv. Drug Deliv. Rev., 58:15-28 (2006)). Mucoadhesive biomaterials have been used to increase the retention time and local concentration of drugs near the apical surface of epithelial cells (Smart et al., Adv. Drug Deliv. Rev., 57:1556-1568 (2005)).

Nanoparticles (NPs) have the potential to make a significant impact on the treatment of many diseases, including cancer, cardiovascular disease, and diabetes. Many NP-based therapeutics are now entering clinical trials or have been approved for use (Davis et al., Nature, 464:1067-1070 (2010); Wang et al., Annu. Rev. Med., 63, 185-198 (2012)), including targeted polymeric nanoparticles (Hrkach et al., Sci. Transl. Med., 4, 128ra39-128ra39 (2012); clinical trial NCT01478893) based on technologies such as that described y Farokhzad et al., Proc. Natl. Acad. Sci. U.S.A., 103:6315-6320 (2006). However, the impact of NPs in the clinic may be limited to a narrow set of indications because NP administration is currently restricted to parenteral methods. Many diseases that could benefit from NP-based therapeutics require frequent administration. Alternate routes of administration, particularly oral, are preferred because of the convenience and compliance by patients (Borner et al., Eur. J. Cancer, 38:349-358(2002)). Intestinal absorption of NPs is highly inefficient because the physicochemical parameters of NPs prevent their transport across cellular barriers such as the intestinal epithelium (Goldberg et al, Nat. Rev. Drug Discov., 2:289-295(2003)).

Many oral NPs have been engineered for uptake by M cells in the Peyer's Patches, although this limits the surface area available for absorption and exposes NPs to underlying immune cells (Shakweh et al., Expert Opin. Drug Deliv., 1:141-163 (2004)). A few NP formulations have targeted cell receptors, but they still suffer from low bioavailability and require high oral drug dosages (Chen et al., Biomaterials, 32:9826-9838 (2011); Jain et al., Nanomed., 7:1311-1337 (2012)). To improve the absorption efficiency of NPs and to make the oral administration of NPs practical in the clinic, new strategies are necessary to overcome the intestinal epithelial barrier.

It is therefore an object of the invention to provide targeted drug delivery nanoparticles with selective receptor mediated delivery through tissue.

It is also an object of the invention to provide drug delivery nanoparticles capable of overcoming the adsorption barriers of conventional drug delivery particles.

It is an additional object of the invention to provide formulations of and methods of using nanoparticle therapeutics that increase patient compliance, reduce systemic toxicity and increase efficacy.

### SUMMARY OF THE INVENTION

Receptor-targeted nanoparticles (“R-NPs”) selectively delivering a therapeutic, prophylactic, or diagnostic agent to tissues expressing the receptor provide enhanced delivery to these tissues by transcytosis. As demonstrated by the examples, FcRn-targeted NPs can be used to deliver a therapeutic such as insulin across the intestinal epithelium. These NPs include a targeting moiety binding an Fc receptor which is covalently or non-covalently bound to the nanoparticle core. The R-targeted nanoparticles can have a variety of particle cores. The R-targeted nanoparticle can contain a polymeric particle core, a lipid particle core, or an inorganic particle core. R nanoparticles can contain hybrid particle cores such as lipid-coated polymeric particles or polymer-coated metal particles. In some embodiments the NPs are formulated in a capsule or pharmaceutically acceptable enteric coated material to facilitate passage through the stomach, into the intestine where the NPs are released and passed through the tissue by transcytosis.

The selectivity of the nanoparticles is determined by the selection of the targeting moieties that bind to receptors on the cells that are being targeted, where the receptors mediate transport into and through the cells. Nanoparticles may include more than one type of receptor, and targeting moieties that bind to ligands other than those mediating transport. Nanoparticles may be formulated for sustained, pulsed or delayed release. Nanoparticles may have targeting moieties mediating initial uptake in a tissue, where additional moieties binding to different receptors are exposed. Nanoparticles may include other binding moieties, such as mucoadhesive ligands, to facilitate retention at the site of uptake.

Representative selective receptors for targeting selective transport through tissue of the nanoparticles include: gp60 and FcRn for delivery to heart, skeletal muscle, or adipose tissue, chorionic gonadotropin receptor, Insulin receptor and insulin-like growth, and Transferrin receptor for delivery to the testis, factor receptor; LDL receptor-related proteins 1 and 2 (LRP1 and LRP2); LDL receptor; Diptheria toxin receptor; Transferrin; Receptor for advanced glycation end products (RAGE); Scavenger receptor (SR); and FcRn for delivery to the brain, receptors for M cells; Terminal galactose (ricin B receptor); aminopeptidase N; pIgA receptor; or Cubulin/Megalin (vitamin B12) for delivery to the intestine, CD23 (for IgE) for delivery to the liver, pIgA and Terminal galactose (ricin B receptor) for delivery to kidney, aminopeptidase N and Megalined for delivery to placenta, FcRn; Transferrin; Terminal galactose (ricin B receptor); pIgA; FcRn; and gp60 for delivery to lung tissue, gp60; aminopeptidase N; and CD23 (for IgE) for delivery to mammary glands, gp60 for delivery to thyroid, and pIgA, Transferrin, Megalin; gp340; and lutropin receptor for delivery to genitourinary tract tissue. In the preferred embodiment, the targeting moieties for the receptors are FcRn.

Methods of making and using R-targeted nanoparticles are provided. R-targeted nanoparticle formulations are provided for the treatment or prevention of diseases or disorders in a subject or patient in need thereof. Examples demonstrate efficacy in controlling blood glucose following oral administration of FcRn-targeted nanoparticles delivering insulin. This establishes that these nanoparticles can pass through tissue, unlike previous nanoparticles, allowing the encapsulated agent to reach the systemic circulation.

### BRIEF DESCRIPTION OF THE DRAWINGS

FIG. 1 is a schematic of Fc-targeted nanoparticle transport across the intestinal epithelium by the FcRn through a transcytosis pathway. (1) IgG Fc on the NP surface binds to the FcRn on the apical side of absorptive epithelial cells under acidic conditions in the intestine. (2) NP-Fc are then trafficked across the epithelial cell through the FcRn transcytosis pathway in acidic endosomes. (3) Upon exocytosis on the basolateral side of the cell, the physiological pH causes IgG Fc to dissociate from the FcRn, and NP-Fc are free to diffuse through the intestinal lamina propria to the capillaries or lacteal and enter systemic circulation.

FIG. 2 is a schematic of R-targeted nanoparticle assembly. NPs consist of a biodegradable PLA core for drug encapsulation and a PEG surface coating for particle stability and to reduce phagocytic uptake. NPs were formed using a nanoprecipitation self-assembly method and surface-modified with IgG Fc for FcRn targeting.

FIG. 3 is a bar graph depicting NP diameter (nm) for non-targeted PLA-PEG nanoparticles and Fc-targeted PLA-PEG nanoparticles. Data are means±SD (n=3).

FIG. 4 is a bar graph depicting the IgG Fc ligand density on the NP surface with (Fc-SH) and without (Fc) thiol modification of the IgG Fc. Data are means±SD (n=3).

FIG. 5 is a bar graph depicting in vitro transepithelial transport of non-targeted NPs, NP-Fc, and NP-Fc with an excess of human IgG Fc as a blocking agent for FcRn. Data are expressed as mean basolateral 3 H disintegrations per minute (DPM) as a percentage of the initial amount of 3 H (±SEM; n=4 wells per group). *P<0.05, two-tailed Student's t-test.

FIG. 6 is a bar graph depicting the relative mFcRn to B-Actin band intensity from Western blot of mouse FcRn (mFcRn) in mouse intestinal tissue.

FIG. 7 is a bar graph depicting the biodistribution of ¹⁴C-labeled non-targeted NPs 1.5 hours, 2.5 hours, 4 hours, 6 hours, and 8 hours after oral administration to fasted wild-type mice. Data are mean % initial dose (ID) per gram of tissue±SEM (n=5 mice per time point).

FIG. 8 is a bar graph depicting the biodistribution of ¹⁴C-labeled Fc-targeted NPs 1.5 hours, 2.5 hours, 4 hours, 6 hours, and 8 hours after oral administration to fasted wild-type mice. Data are mean % initial dose (ID) per gram of tissue±SEM (n=5 mice per time point).

FIG. 9 is a graph depicting release of ¹⁴C from ¹⁴C-labeled NPs in PBS at 37° C. Data are means±SD for n=4 release experiments.

FIG. 10 is a graph depicting the total absorbed ¹⁴C over time for non-targeted NPs and NP-Fc after administration by oral gavage. Data are mean % ID measured in all of the organs added together±SEM (n=5 mice per time point). **P<0.01 for comparison of non-targeted NPs and NP-Fc at respective time point, two-tailed Student's t-test.

FIG. 11 is a graph depicting the insulin release from insulin loaded Fc-targeted PLA-PEG nanoparticles in PBS buffer. Data are means±SD (n=3 per time point).

FIG. 12 is a graph depicting the blood glucose response of fasted wild-type mice to free insulin and to insulin encapsulated and released from Fc-targeted PLA-PEG nanoparticles prior to administration. Fasted wild-type mice received the insulin (3.3 U/kg) administered by tail-vein injection. Data are means±SEM (n=3 mice per group).

FIG. 13 is a graph depicting the blood glucose response of fasted wild-type mice to free insulin solution, Fc-targeted PLA-PEG nanoparticles containing no insulin, non-targeted PLA-PEG nanoparticles containing insulin, and Fc-targeted PLA-PEG nanoparticles containing insulin, each administered by oral gavage. Data are means±SEM (n=6 mice per group). *P<0.05 for comparison of non-targeted and Fc-targeted insulin nanoparticles at corresponding time points, two-tailed Student's t-test.

FIG. 14 is a graph depicting the blood glucose response of fasted wild-type mice to IgG Fc-targeted PLA-PEG nanoparticles containing insulin, IgG Fc-targeted PLA-PEG nanoparticles containing insulin administered concurrently with excess of IgG Fc, and chicken IgY Fc-targeted nanoparticles containing insulin, each administered by oral gavage. Data are means±SEM (n=5 mice per group). **P<0.01 for comparison between insNP-Fc with insNP-Fc+free IgG Fc at the 15 and 19 h timepoints and between insNP-IgG Fc and insNP-IgY Fc at the 10, 15, and 19 h timepoints using a two-tailed Student's t-test.

FIG. 15 is a graph depicting the blood glucose response to equivalent insulin doses (3.3 U/kg) administered by tail-vein injection into fasted wild-type and FcRn KO mice. Data are means±SEM (n=3 mice per group).

FIG. 16 is a graph depicting the blood glucose response of fasted FcRn KO mice to free insulin solution, NP-Fc containing no insulin, non-targeted insNP, or insNP-Fc, each administered by oral gavage. Data are means±SEM (n=5 mice per group).

FIG. 17 is a graph depicting the blood glucose response of fasted wild-type and FcRn KO mice dosed by oral gavage with non-targeted insNP and insNP-Fc at two different doses. *P<0.05 for comparison between insNP-Fc at 1.1 U/kg and each of the other groups at corresponding timepoints, two-tailed Student's t-test.

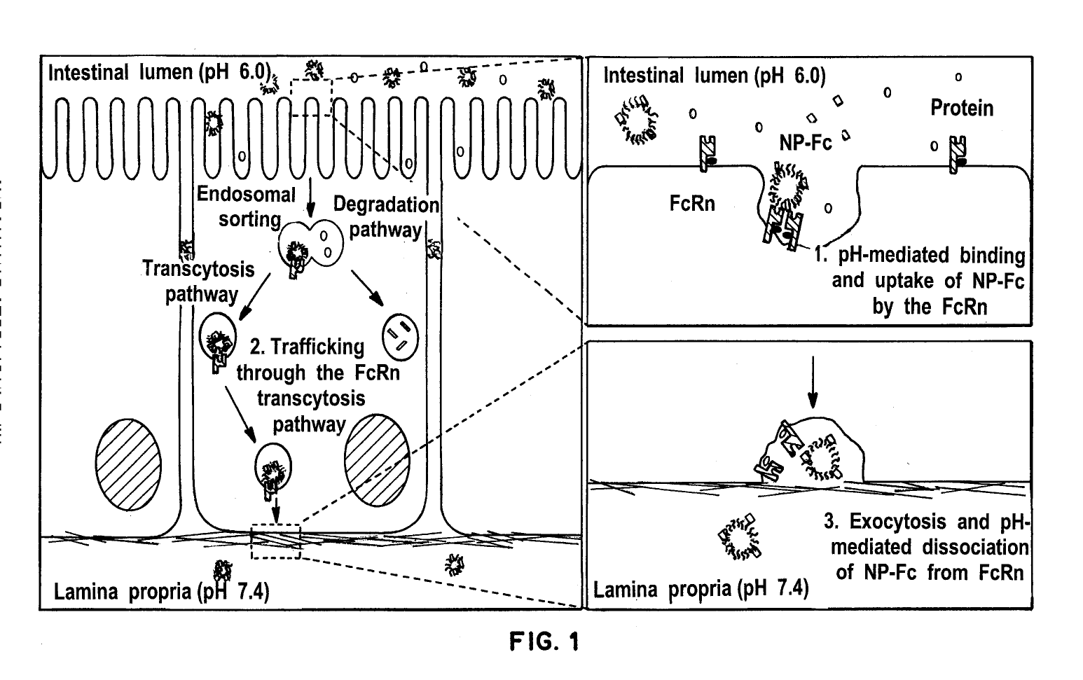

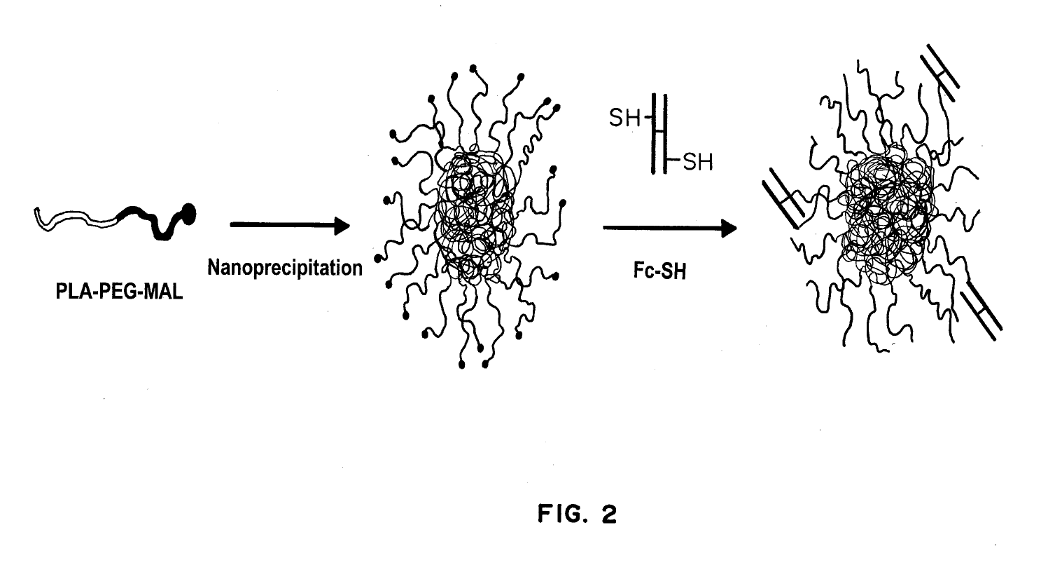

### DETAILED DESCRIPTION OF THE INVENTION

R-targeted nanoparticles containing a therapeutic, prophylactic, and/or diagnostic agent provide selective receptor mediated delivery the therapeutic, prophylactic, or diagnostic agent into cells expressing the receptor. The R-targeted nanoparticles can be delivered systemically or locally, topically, orally, mucosally or by direct injection into tissue including the cells expressing the receptor, such as a tumor.

### I. Definitions

The terms “treating” or “preventing”, as used herein, can include preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The terms “bioactive agent” and “active agent”, as used interchangeably herein, include physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The terms “sufficient” and “effective”, as used interchangeably herein, refer to an amount (e.g. mass, volume, dosage, concentration, and/or time period) needed to achieve one or more desired result(s).

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier”, as used herein, refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “small molecule”, as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties.

The term “mean particle size”, as used herein, generally refers to the statistical mean particle size (diameter) of the particles in the composition. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering. Two populations can be the to have a “substantially equivalent mean particle size” when the statistical mean particle size of the first population of nanoparticles is within 20% of the statistical mean particle size of the second population of nanoparticles; more preferably within 15%, most preferably within 10%.

The terms “monodisperse” and “homogeneous size distribution”, as used interchangeably herein, describe a population of particles, nanoparticles, or nanoparticles all having the same or nearly the same size. As used herein, a monodisperse distribution refers to particle distributions in which 90% of the distribution lies within 5% of the mean particle size.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. The targeting moiety or a sufficient plurality of targeting moieties may be used to direct the localization of a particle or an active entity. The active entity may be useful for therapeutic, prophylactic, or diagnostic purposes.

The term “mediated transport” refers to transport mediated by a membrane transport protein. There are three types of mediated transport: uniport, symport, and antiport. A uniporter is an integral membrane protein that is involved in facilitated diffusion. They can be either a channel or a carrier protein. Uniporter carrier proteins work by binding to one molecule of solute at a time and transporting it with the solute gradient. Uniporter channels open in response to a stimulus and allow the free flow of specific molecules. Uniporters may not utilize energy other than the solute gradient. Thus they may only transport molecules with the solute gradient, and not against it.

A symporter is an integral membrane protein that is involved in movement of two or more different molecules or ions across a phospholipid membrane such as the plasma membrane in the same direction, and is, therefore, a type of cotransporter. Typically, the ion(s) will move down the electrochemical gradient, allowing the other molecule(s) to move against the concentration gradient. The movement of the ion(s) across the membrane is facilitated diffusion, and is coupled with the active transport of the molecule(s). Although two or more types of molecule are transported, there may be several molecules transported of each type.

An antiporter (also called exchanger or counter-transporter) is an integral membrane protein involved in secondary active transport of two or more different molecules or ions (i.e., solutes) across a phospholipid membrane such as the plasma membrane in opposite directions. In secondary active transport, one species of solute moves along its electrochemical gradient, allowing a different species to move against its own electrochemical gradient. This movement is in contrast to primary active transport, in which all solutes are moved against their concentration gradients, fueled by ATP.

The term “transcytosis” refers to a mechanism for transcellular transport in which a cell encloses extracellular material in an invagination of the cell membrane to form a vesicle, then moves the vesicle across the cell to eject the material through the opposite cell membrane by the reverse process. This is also called vesicular transport.

The term “endocytosis” refers to the uptake by a cell of material from the environment by invagination of its plasma membrane and includes both phagocytosis and pinocytosis.

### II. Receptor-Targeted Nanoparticles

R-targeted nanoparticles containing a particle core and a plurality of receptor-targeting moieties are provided for the delivery of therapeutic, prophylactic, and/or diagnostic agents. The R-targeted nanoparticles are capable of being actively transported via transcytosis into and through the cells expressing the receptor. This provides a means of moving nanoparticles into tissue and the circulation when they would normally be unable to do so, as well as through barriers such as the blood brain barrier.

A. Particle Core

The R-targeted nanoparticles contain a particle core. The particle core can be a polymeric particle, a lipid particle, a solid lipid particle, an inorganic particle, or combinations thereof. For example, the particle core can be a lipid-stabilized polymeric particle. In preferred embodiments the particle core is a polymeric particle, a solid lipid particle, or a lipid-stabilized polymeric particle, preferably a polymeric particle.

The particle core may have any diameter. The particle core can have a diameter of about 10 nm to about 10 microns, about 10 nm to about 1 micron, about 10 nm to about 500 nm, about 20 nm to about 500 nm, or about 25 nm to about 250 nm. In preferred embodiments the particle core is a nanoparticle core having a diameter from about 25 nm to about 250 nm. In the most preferred embodiment the particles have a diameter of three to 150 nm.

The particle core may have any zeta potential. particle core can have a zeta potential from −300 mV to +300 mV, −100 mV to +100 mV, from −50 mV to +50 mV, from −40 mV to +40 mV, from −30 mV to +30 mV, from −20 mV to +20 mV, from −10 mV to +10 mV, or from −5 mV to +5 mV. The particle core can have a negative zeta potential. The particle core can have a positive zeta potential. In some embodiments the particle core has a substantially neutral zeta potential, i.e. the zeta potential is approximately 0 mV. In preferred embodiments the particle core has a zeta potential of approximately −20 mV to +20 mV, more preferably −10 mV to +10 mV.

i. Polymeric Particle Core

The particle core can be a polymeric particle core. The polymeric particle core can be formed from biodegradable polymers, non-biodegradable polymers, or a combination thereof. The polymeric particle core can be a biodegradable polymeric core in whole or in part. For example, an imaging agent or diagnostic agent that needs to be retained in the particles and cleared from the body can be encapsulated in a non-biodegradable polymer matrix.

Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water. Representative biodegradable polymers include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. Non-biodegradable polymers can include ethylene vinyl acetate, poly(meth) acrylic acid, polyamides, copolymers and mixtures thereof.

Excipients may also be added to the core polymer to alter its porosity, permeability, and or degradation profile.

The polymeric core can contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In preferred embodiments, the polymeric core contains biodegradable polyesters or polyanhydrides such as poly(lactic acid), poly(glycolic acid), and poly(lactic-co-glycolic acid).

The molecular weight of the hydrophobic polymer can be varied to tailor the properties of polymeric particle core. For example, the molecular weight of the hydrophobic polymer segment can be varied to engineer nanoparticles possessing the required average particle size and degradation profile. The hydrophobic polymer segment has a molecular weight of between about 150 Da and about 100 kDa, more preferably between about 1 kDa and about 75 kDa, most preferably between about 5 kDa and about 50 kDa.

The polymeric particle core can contain an amphiphilic polymer. Amphiphilic polymers can include block copolymers of any of the hydrophobic and hydrophilic polymers described above. In some embodiments the amphiphilic polymer is a copolymer containing a hydrophobic polyhydroxyacid block and a hydrophilic polyalkylene glycol block. The amphiphilic polymer can be a PLGA-PEG block copolymer, and PGA-PEG block copolymer, or a PLGA-PEG block copolymer.

PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, or by ring opening polymerization techniques (ROMP).

Copolymers containing poly(ester-ether)s, e.g., polymers having repeat units joined by ester bonds (e.g., R—C(O)—O—R′ bonds) and ether bonds (e.g., R—O—R′ bonds) may be formed as a hydrolyzable polymer, containing carboxylic acid groups, conjugated with poly(ethylene glycol) repeat units to form a poly(ester-ether).

The polymeric particle core can contain any of the above polymers or blends or copolymers thereof. The polymeric particle core can contain one, two, three, or more different polymers.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

The amphiphilic lipid can have a molecular weight of 200 to 1000, e.g., 700-900. By containing a relatively small amount of lipid, the nanoparticles avoid the negative impact that a tri, tetra or higher layer of lipid could have on a nanoparticle, such as an adverse effect on drug release. Thus, in one embodiment, the nanoparticles comprise approximately 10% to 40% lipid (by weight), and will have a size of about 90 nm to about 40 nm in diameter.

In a particular embodiment, an amphiphilic component that can be used to form an amphiphilic layer is lecithin, and, in particular, phosphatidylcholine. Lecithin forms a phospholipid bilayer having the hydrophilic (polar) heads facing aqueous solutions, and the hydrophobic tails facing each other. Lecithin has an advantage of being a natural lipid that is available from, e.g., soybean, and already has FDA approval for use in other delivery devices.

The particle core can be a lipid particle core. In some embodiments the particle core is a lipid nanoparticle. Lipid particles and lipid nanoparticles are known in the art. The lipid particles and lipid nanoparticles can be lipid micelles, liposomes, or solid lipid particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle core can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents. The particle core can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle core can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy) propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1, 2-dialkyloxy-3-dimethylammonium propanes, dioctadecylamidoglycylspermine (DOGS), 3-[N-(N′,N′-dimethylamino-ethane) carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy) ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy) ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

The particle core can be an inorganic particle such as metal or semiconductor particles. The particle core can be a metal nanoparticle, a semiconductor nanoparticle, or a core-shell nanoparticle. Inorganic particles and inorganic nanoparticles can be formulated into a variety of shapes such as rods, shells, spheres, and cones. The inorganic particle may have any dimension. The inorganic particle can have a greatest dimension less than 1 micron, from about 10 nm to about 1 micron, from about 10 nm to about 500 nm, or from 10 nm to about 250 nm.

The inorganic particle core can contain a metal. Suitable metals can include alkali metals such as lithium, sodium, potassium, rubidium, cesium and francium; alkaline earth metals such as beryllium, magnesium, calcium, strontium, barium and radium; transition metals such as zinc, molybdenum, cadmium, scandium, titanium, vanadium, chromium, manganese, iron, cobalt, nickel, copper, yttrium, zirconium, niobium, technetium, ruthenium, rhodium, palladium, silver, tungsten, iridium, and platinum; post-transition metals such as aluminum, gallium, indium, tin, thallium, lead, and bismuth; lanthanoids such as lanthanum, cerium, neodymium, and europium; and actinoids such as actinium, thorium, protactinium, uranium, neptunium, and plutonium. The metal can be biodegradable or non-biodegradable. Biodegradable metals can include alloys of iron or magnesium with the above metals, including alloys of magnesium, aluminum, and zinc.

The inorganic particle core can contain a metal oxide. Metal oxides of any of the above metals are contemplated. Suitable metal oxides can include metal oxides that contain one or more of the following metals: titanium, scandium, iron, tantalum, cobalt, chromium, manganese, platinum, iridium, niobium, vanadium, zirconium, tungsten, rhodium, ruthenium, copper, zinc, yttrium, molybdenum, technetium, palladium, cadmium, hafnium, rhenium and combinations thereof. Suitable metal oxides can include cerium oxides, platinum oxides, yttrium oxides, tantalum oxides, titanium oxides, zinc oxides, iron oxides, magnesium oxides, aluminum oxides, iridium oxides, niobium oxides, zirconium oxides, tungsten oxides, rhodium oxides, ruthenium oxides, alumina, zirconia, silicone oxides such as silica based glasses and silicon dioxide, or combinations thereof. The metal oxide can be non-biodegradable. The metal oxide can be a biodegradable metal oxide. Biodegradable metal oxides can include silicon oxide, aluminum oxide and zinc oxide.

The particle core can be a hybrid particle. Hybrid particle, as used herein, refers to a particle that combines the features of two or more of polymeric particles, lipid particles, and inorganic particles. Examples of hybrid particles can include polymer-stabilized liposomes, polymer-coated inorganic particles, or lipid-coated polymeric particles. The hybrid particle can contain a polymeric inner region, a lipid inner region, or an inorganic inner region. The hybrid particle can contain a polymer outer layer, a lipid outer layer, or an inorganic outer layer.

The particle core can be a polymer-stabilized lipid particle. The particle core can be a polymer-stabilized liposome. Polymer-stabilized liposomes are described, for example, in WO 2008/082721 by Dominguez et al. The particle core can be a polymer-stabilized solid lipid particle. Solid lipid particles have been coated with polymers to impart stability (see Nahire et al., Biomacromolecules, 14:841-853 (2013)) or to impart stealth properties (see Uner and Yener, Int. J. Nanomedicine, 2:289-300 (2007)). The polymer-stabilized liposomes and polymer-stabilized solid lipid particles include a lipid particle core stabilized by the presence of a coating polymer. The coating polymer can be covalently or non-covalently bound to the lipid particle. The coating polymer can be a lipophilic polymer, a biodegradable polymer, a polymer decreasing uptake by the RES, or a combination thereof.

The particle core can be a polymer-stabilized inorganic particle such as a polymer-coated metal nanoparticle. WO 2013/070653 by Alocilja et al. described metal nanoparticle stabilized by a polysaccharide coating polymer.

Suitable lipophilic polymers can include aliphatic polyesters, such as polylactic acid, polyglycolic acid and their copolymers; poly(ε-caprolactone), poly(δ-valerolactone), polyesters with longer (i.e., Ci5 to C25) hydrocarbon chains; dendritic polymers of polyesters containing a modified terminal hydroxyl; aliphatic and aromatic polycarbonates; aliphatic polyamides, polypeptides; polyesteramides; polyurethanes; silicones, such as poly(dimethylsyloxanes); lipophilic poly(phosphazenes); poly(methacrylic acid), poly(styrene) and hydrophobic polyacrylic, polyvinyl and polystyrene carriers.

B. Transport Mediating Receptors

Transport mediating receptors are known and can be used to target the NPs for uptake. Some of these are tissue specific, and can therefore be used to provide selective uptake predominantly into a targeted tissue.

These may be proteins, peptides, amino acids, nucleic acid molecules, small molecules, lipids, carbohydrate, or combinations thereof.

TABLE 1
Trancytosis Receptors:

| Organ | Receptor |
|---|---|
| Heart, skeletal muscle, | gp60 or FcRn |
| adipose tissue: | |
| Testis: | Chorionic gonadotropin receptor, |
| | Insulin receptor and insulin-like |
| | growth, FcRn, and Transferrin receptor |
| Brain: | Insulin receptor, insulin-like growth |
| | factor receptor; LDL receptor-related |
| | proteins 1 and 2 (LRP1 and LRP2); |
| | LDL receptor; Diptheria toxin |
| | receptor; Transferrin; Receptor for |
| | advanced glycation end products |
| | (RAGE); Scavenger receptor (SR); |
| | FcRn |
| Intestine: | M cells; Terminal galactose (ricin B |
| | receptor); aminopeptidase N; pIgA |
| | receptor; Cubulin/Megalin (vitamin |
| | B12); FcRn; CD23 (for IgE) |
| Liver: | pIgA receptor, FcRn |
| Kidney: | pIgA; Terminal galactose (ricin B |
| | receptor); Megalin receptor, FcRn |
| Placenta: | aminopeptidase N; Megalin; FcRn, |
| | pIgA receptor, transferrin |
| Lungs: | Transferrin; Terminal galactose (ricin |
| | B receptor); pIgA; FcRn; gp60; CD23 |
| | (for IgE) |
| Mammary Gland: | gp60; aminopeptidase N; pIgA; FcRn; |
| | Transferrin |
| Thyroid: | gp60; megalin |
| Genitourinary: | pIgA: Transferrin: Megalin; gp340; |
| | lutropin receptor; FcRn |

FcRn is used as an exemplary receptor to demonstrate targeting of Nps for tissue selective endocytoic mediated drug delivery. FcRn has been shown to mediate the transcytosis of IgG across several epithelial and endothelial barriers (Kuo et al., MAbs 3:422-430 (2011). Harnessing the transcytosis pathway to cross the intestinal epithelium offers the advantage of leaving intact the integrity of the epithelial barrier, avoiding potential safety issues and adverse effects associated with manipulating the permeability of the intestine for paracellular or transcellular transport. An additional advantage of targeting the FcRn is that this receptor is expressed throughout the intestine, providing a significant increase in the available absorption surface area for NP-Fc, which is in contrast with other drug delivery systems that target only a specific portion of the intestine such as the Peyer's patches (Goldberg, et al., Nat. Rev. Drug Discov. 2:289-295 (2003)).

The Fc receptor FcRn (n for neonatal) was first identified in 1970s as a protein that mediates transfer of maternal, milk born IgGs across the rodent neonatal intestine. More recent data have indicated that it not only delivers IgG across the maternofetal barrier during gestation, but is also responsible for the maintenance of serum IgGs level to provide humoral immunity during the first weeks of independent life. The IgG transfer is highly selective and is thought to involve specific receptors that bind to the Fc region of the IgG molecule.

The nature of FcRn and its interaction with IgGs involved in the transport of the IgG across subcell layer has been well characterized. In humans, maternal IgG is actively transported across the placenta. Several IgG-binding proteins have been isolated from placenta. Fc.gamma.RII was detected in placental endothelium and Fc.gamma.RIII in syncytiotrophoblasts. Both of these receptors, however, showed a relatively low affinity for monomeric IgG. The isolation from placenta of a cDNA encoding a human homolog of the rat and mouse enterocyte receptor for IgG has been reported (Story et al., J. Exp. Med., 180:2377 (1994)). FcRn has been reported to be present in endothelial cells of human muscle vasculature. In placental endothelial cells, FcRn is responsible for selective and controlled transport of IgG, as a result of which the fetal humoral immunity is ensured.

Table 1 provides a list of representative receptors and tissues that express the receptors. Receptors can also be used to target nanoparticles to the liver, the biliary system, into and out of the eye through the cornea and into the bloodstream, the brain, CNS, skin/sebaceous glands, genitourinary tract, and the vagina or rectum.

The FcRn is used herein as an exemplary receptor used to target the NPs for selective uptake by cells expressing the receptor. The Fc-targeted nanoparticles contain a targeting moiety that binds the neonatal Fc receptor (FcRn). Any FcRn binding moiety can be used as an Fc-targeting moiety in an Fc-targeted nanoparticle. In some embodiments, after a Fc-targeted nanoparticle (e.g., containing an Fc fragment) is delivered to the interior of a cell, the FcRn targeting moiety (e.g., Fc fragment) can target the drug delivery system to immune system components (e.g. macrophages). In some embodiments, the targeting moiety on an Fc-targeted nanoparticle may be shed once the drug delivery system has crossed the epithelium. This shedding of the FcRn binding moiety may be accomplished using a cleavable linker that associates the FcRn binding moiety with the drug delivery system.

In some embodiments, a receptor binding moiety may be associated with the R-targeted nanoparticle via a cleavable linker, such as chemically-responsive linkers, pH-responsive linkers, heat-responsive linkers, light-responsive linkers (e.g., linkers that are cleaved in response to ultraviolet light), etc.

For example, the linker may be a protease-cleavable linker. Exemplary linkers include peptide linkers, esterase-sensitive linkers, disulfide linkers, and protease-sensitive linkers. In some embodiments, a receptor binding moiety may not be shed from the R-targeted nanoparticle at any point during or after drug delivery. For example, the linker may comprise the recognition sequence for matrix metalloproteinases (MMPs) that are typically either secreted into the extracellular space or bound to the external surface of a plasma membrane. When the drug R-targeted nanoparticle reaches a cellular target, it is exposed to extracellular MMPs, which act upon the cleavable linker and shed the Fc fragment.

Additional targeting moieties may help direct drug delivery systems to their appropriate targets after crossing the intestinal epithelium, for example, additional targeting moieties may target components of the extracellular matrix (ECM). In some embodiments, it may be desirable to target a drug delivery system to the ECM because it can minimize contact of the drug delivery system with cells of the immune system. For example, an additional targeting moiety may target collagen IV, one of the most abundant proteins of the basal lamina of the ECM.

One of ordinary skill in the art will recognize that any additional targeting moiety which directs the drug delivery system to any target site may be utilized. Exemplary additional targeting moieties include, but are not limited to, proteins (e.g., peptides, antibodies, glycoproteins, polypeptides, etc., or characteristic portions thereof), nucleic acids (e.g. aptamers, Spiegelmers, RNAi-inducing entities, etc., or characteristic portions thereof), carbohydrates (e.g. monosaccharides, disaccharides, polysaccharides, etc., or characteristic portions thereof), lipids or characteristic portions thereof, small molecules or characteristic portions thereof, viruses, nanoparticles, etc., as described herein.

Any FcRn binding moiety may be used as a targeting moiety in R-targeted nanoparticle. An FcRn binding moiety means any entity (e.g., peptides, glycopeptides, proteins, glycoproteins, polynucleotides, aptamers, spiegelmers, antibodies (e.g., monoclonal antibodies), antibody fragments, small molecule ligands, carbohydrate ligands, nanobodies, avimers, metal complexes, etc.) that can be specifically bound by the FcRn receptor with subsequent active transport by the FcRn receptor of the FcRn binding moiety and the particle. Although described herein with reference to the FcRN receptor and binding moiety, it is understood that the following comments are applicable to other receptors useful in targeting nanoparticles for tissue transport by transcytosis, including those listed in Table 1.

The FcRn receptor has been isolated from several mammalian species, including humans. The sequence of the human FcRn, rat FcRn, and mouse FcRn may be found in Story et al. (1994, J. Exp. Med., 180:2377). The FcRn receptor molecule is well characterized. The FcRn receptor binds IgG (but not other immunoglobulin classes such as IgA, IgD, IgM and IgE) at a relatively low pH, actively transports the IgG transcellularly in a luminal to serosal direction, and then releases the IgG at the relatively high pH found in the interstitial fluids. As will be recognized by those of ordinary skill in the art, FcRn receptors can be isolated by cloning or by affinity purification using, for example, monoclonal antibodies. Such isolated FcRn receptors then can be used to identify and isolate FcRn binding moieties. The FcRn binding moiety can be a small molecule, a protein or peptide, an immunoglobulin, a glycoprotein, a polynucleotide (e.g., aptamer, RNAi-inducing entity, etc.), a carbohydrate, a lipid, or any other type of chemical compound. In certain embodiments, the FcRn binding moiety is a protein or peptide. In some embodiments, the FcRn binding moiety is an immunoglobulin (e.g. Fc fragment). In some embodiments, it is an aptamer. In some embodiments, it is a spiegelmer. In some embodiments, it is an RNAi-inducing entity (e.g., siRNA, shRNA, miRNA, etc.). In some embodiments, the binding moiety is a small molecule.

In certain embodiments, an FcRn binding moiety is an Fc fragment. In certain embodiments, an FcRn binding moiety is an Fc fragment of an IgG antibody. In some embodiments, an FcRn binding moiety is an Fc fragment of any isotype of IgG antibody (e.g., IgG 1, IgG 2, IgG 2a, IgG 2b, IgG 3, IgG 4, etc.).

In some embodiments, the sequence of the Fc portion of a human IgG 1 antibody is as follows:

(SEQ ID NO.: 1)
TCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKG
FYPSDIAVEWESNGQPENNYKTTPPVLDSDGPFFLYSKLTVDKSRWQQG
NVFSCSVMHEALHNHYTQKSLSLSPGK.

In some embodiments, the sequence of the Fc portion of a human IgG 1 antibody is as follows:

(SEQ ID NO.: 2)
ZVQLEQSGPGLVRPSQTLSLTCTVSGTSFDDYYWTWVRQPPGRGLEWIG
YVFYTGTTLLDPSLRGRVTMLVNTSKNQFSLRLSSVTAADTAVYYCARN
LIAGGIDVWGQGSLVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKKVEPKSC

In some embodiments, the nucleic acid sequence corresponding to the Fc portion of a human IgG 1 antibody is as follows:

(SEQ ID NO.: 3)
GACAAAACTCACACATGCCCACCGTGCCCAGCACCTGAACTCCTGGGGG
GACCGTCAGTCTTCCTCTTCCCCCCAAAACCCAAGGACACCCTCATGAT
CTCCCGGACCCCTGAGGTCACATGCGTGGTGGTGGACGTGAGCCACGAA
GACCCTGAGGTCAAGTTCAACTGGTACGTGGACGGCGTGGAGGTGCATA
ATGCCAAGACAAAGCCGCGGGAGGAGCAGTACAACAGCACGTACCGTGT
GGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAA
CCATCTCCAAAGCCAAAGGGCAGCCCCGAGAACCACAGGTGTACACCCT
GCCCCCATCCCGGGAGGAGATGACCAAGAACCAGGTCAGCCTGACCTGC
CTGGTCAAAGGCTTCTATCCCAGCGACATCGCCGTGGAGTGGGAGAGCA
ATGGGCAGCCGGAGAACAACTACAAGACCACGCCTCCCGTGCTGGACTC
CGACGGCTCCTTCTTCCTCTACAGCAAGCTCACCGTGGACAAGAGCAGG
TGGCAGCAGGGGAACGTCTTCTCATGCTCCGTGATGCATGAGGGTCTGC
ACAACCACTACACGCAGAAGAGCCTCTCCCTGTCTCCGGGTAAATGA

In some embodiments, the nucleic acid sequence corresponding to the Fc portion of a human IgG 2 antibody is as follows:

(SEQ ID NO.: 4)
GTGGAGTGCCCACCTTGCCCAGCACCACCTGTGGCAGGACCTTCAGTCT
TCCTCTTCCCCCCAAAACCCAAGGACACCCTGATGATCTCCAGAACCCC
TGAGGTCACGTGCGTGGTGGTGGACGTGAGCCACGAAGACCCCGAGGTC
CAGTTCAACTGGTACGTGGACGGCATGGAGGTGCATAATGCCAAGACAA
AGCCACGGGAGGAGCAGTTCAACAGCACGTTCCGTGTGGTCAGCGTCCT
CACCGTCGTGCACCAGGACTGGCTGAACGGCAAGGAGTACAAGTGCAAG
GTCTCCAACAAAGGCCTCCCAGCCCCCATCGAGAAAACCATCTCCAAAA
CCAAAGGGCAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAAGACCACACCTCCCATGCTGGACTCCGACGGCTCCTT
CTTCCTCTACAGCAAGCTCACCGTGGACAAGAGCAGGTGGCAGCAGGGG
AACGTCTTCTCATGCTCCGTGATGCATGAGGCTCTGCACAACCACTACA
CACAGAAGAGCCTCTCCCTGTCTCCGGGTAAATGAGTGCCACGGCTAGC
TGG.

In some embodiments, the nucleic acid sequence corresponding to the Fc portion of a human IgG 3 antibody is as follows:

(SEQ ID NO.: 5)
GACACACCTCCCCCGTGCCCAAGGTGCCCAGCACCTGAACTCCTGGGAG
GACCGTCAGTCTTCCTCTTCCCCCCAAAACCCAAGGATACCCTTATGAT
TTCCCGGACCCCTGAGGTCACGTGCGTGGTGGTGGACGTGAGCCACGAA
GACCCCGAGGTCCAGTTCAAGTGGTACGTGGACGGCGTGGAGGTGCATA
ATGCCAAGACAAAGCCGCGGGAGGAGCAGTTCAACAGCACGTTCCGTGT
GGTCAGCGTCCTCACCGTCCTGCACCAGGACTGGCTGAACGGCAAGGAG
TACAAGTGCAAGGTCTCCAACAAAGCCCTCCCAGCCCCCATCGAGAAAA
CCATCTCCAAAACCAAAGGACAGCCCCGAGAACCACAGGTGTACACCCT
GCCCCCATCCCGGGAGGAGATGACCAAGAACCAGGTCAGCCTGACCTGC
CTGGTCAAAGGCTTCTACCCCAGCGACATCGCCGTGGAGTGGGAGAGCA
GCGGGCAGCCGGAGAACAACTACAACACCACGCCTCCCATGCTGGACTC
CGACGGCTCCTTCTTCCTCTACAGCAAGCTCACCGTGGACAAGAGCAGG
TGGCAGCAGGGGAACATCTTCTCATGCTCCGTGATGCATGAGGCTCTGC
ACAACCGCTTCACGCAGAAGAGCCTCTCCCTGTCTCCGGGTAAATGA.

In some embodiments, the nucleic acid sequence corresponding to the Fc portion of a human IgG 4 antibody is as follows:

(SEQ ID NO.: 6)
CCCCCATGCCCATCATGCCCAGCACCTGAGTTCCTGGGGGGACCATCAG
TCTTCCTGTTCCCCCCAAAACCCAAGGACACTCTCATGATCTCCCGGAC
CCCTGAGGTCACGTGCGTGGTGGTGGACGTGAGCCAGGAAGACCCCGAG
GTCCAGTTCAACTGGTACGTGGATGGCGTGGAGGTGCATAATGCCAAGA
CAAAGCCGCGGGAGGAGCAGTTCAACAGCACGTACCGTGTGGTCAGCGT
CCTCACCGTCCTGCACCAGGACTGGCTGAACGGCAAGGAGTACAAGTGC
AAGGTCTCCAACAAAGGCCTCCCGTCCTCCATCGAGAAAACCATCTCCA
AAGCCAAAGGGCAGCCCCGAGAGCCACAGGTGTACACCCTGCCCCCATC
CCAGGAGGAGATGACCAAGAACCAGGTCAGCCTGACCTGCCTGGTCAAA
GGCTTCTACCCCAGCGACATCGCCGTGGAGTGGGAGAGCAATGGGCAGC
CGGAGAACAACTACAAGACCACGCCTCCCGTGCTGGACTCCGACGGCTC
CTTCTTCCTCTACAGCAGGCTAACCGTGGACAAGAGCAGGTGGCAGGAG
GGGAATGTCTTCTCATGCTCCGTGATGCATGAGGCTCTGCACAACCACT
ACACACAGAAGAGCCTCTCCCTGTCTCCGGGTAAATG.

An FcRn binding moiety can be at least 50% homologous to any sequence of an Fc portion of any IgG antibody. In some embodiments, an FcRn binding moiety can be at least 60% homologous to any sequence of an Fc portion of any IgG antibody. In certain embodiments, an FcRn binding moiety can be at least 70% homologous to any sequence of an Fc portion of any IgG antibody. In certain embodiments, an FcRn binding moiety can be at least 80% homologous to any sequence of an Fc portion of any IgG antibody. In certain embodiments, an FcRn binding moiety can be at least 90% homologous to any sequence of an Fc portion of any IgG antibody. In certain embodiments, an FcRn binding moiety can be at least 95% or at least 98% homologous to any sequence of an Fc portion of any IgG antibody.

An FcRn binding moiety can at least 50% homologous to any of SEQ ID NOs.: 1-6. In certain embodiments, an FcRn binding moiety can be at least 60% homologous to any of SEQ ID NOs.: 1-6. In certain embodiments, an FcRn binding moiety can be at least 70% homologous to any of SEQ ID NOs.: 1-6. In certain embodiments, an FcRn binding moiety can be at least 80% homologous to any of SEQ ID NOs.: 1-6. In certain embodiments, an FcRn binding moiety can be at least 90% homologous to any of SEQ ID NOs.: 1-6. In certain embodiments, an FcRn binding moiety can be at least 95% or at least 98% homologous to any of SEQ ID NOs.: 1-6.

In some embodiments, an FcRn binding moiety may contain any portion of the Fc fragment of any IgG isotype. In specific embodiments, the portion of the Fc fragment retains the ability to bind the FcRn receptor. In some embodiments, an FcRn binding moiety may be any substance that is able to specifically bind to the FcRn receptor. In some embodiments, an FcRn binding moiety is any substance that is able to bind to the FcRn receptor with an equilibrium dissociation constant, K_{d}, that is 10⁻³ M or less, 10⁻⁴ M or less, 10⁻⁵ M or less, 10⁻⁶ M or less, 10⁻⁷ M or less, 10-8 M or less, 10⁻⁹ M or less, 10⁻¹⁰ M or less, 10⁻¹¹ M or less, or 10⁻¹² M or less under the conditions employed.

In some embodiments, an FcRn binding moiety may be modified such that it is less immunogenic than the unmodified FcRn binding moiety. In some embodiments, an Fc binding moiety may be modified such that it does not bind to complement systems.

In certain embodiments, rather than using an FcRn binding moiety, the polymeric drug delivery system is conjugated to a binding moiety of another receptor (i.e., a non-FcRn receptor) found on endothelial or epithelial cells. In certain particular embodiments, the non-FcRn receptor is found on endothelial cells. In some embodiments, the non-FcRn receptor is found on epithelial cells. In certain embodiments, the polymeric drug delivery system is conjugated not only to an FcRn binding moiety but also a binding moiety for another receptor found on endothelial or epithelial cells. In certain embodiments, the binding moiety of a non-FcRn receptor is a binding moiety of an adhesion molecule (e.g., selectins, integrins, immunoglobulin superfamily, cadherins, etc.). For a list of adhesion molecules, see, e.g., Carlos et al. (1994, Blood, 84:2068; incorporated herein by reference). In certain embodiments, the other binding moiety is a binding moiety of a member of the immunoglobulin superfamily (e.g., NCAM-1; ICAM-1; ICAM-2; LFA-3; major histocompatibility complex molecules (MHCs), particular class I MHC; PECAM (CD31); VCAM-1; MAdCAM-1; PECAM-1). In certain embodiments, the other binding moiety is a binding moiety of vascular cell adhesion molecule (e.g., VCAM-1). In certain specific embodiments, the polymeric drug delivery system has conjugated to it an Fc fragment and a binding moiety of VCAM-1. In certain embodiments, the other binding moiety is a binding moiety of intercellular adhesion molecule (e.g., ICAM-1, ICAM-2). In certain specific embodiments, the polymeric drug delivery system has conjugated to it an Fc fragment and a binding moiety of an ICAM receptor (e.g., ICAM-1, ICAM-2). In some embodiments, the other binding moiety is a binding moiety of selectin (e.g., E-selectin, P-selectin, L-selectin). In certain specific embodiments, the polymeric drug delivery system has conjugated to it an Fc fragment and a binding moiety of selectin (e.g., E-selectin, P-selectin, L-selectin). In some embodiments, the other binding moiety is a binding moiety of a member of the integrin family or has conjugated to it an Fc fragment and a binding moiety of a member of the integrin family In some embodiments, the other binding moiety is a binding moiety of a member of the cadherin family (e.g., cadherin E, cadherin P, cadherin VE (CD144), desmocollin 2, desmoglein 2, etc.). In certain embodiments, the polymeric drug delivery system has conjugated to it an Fc fragment and a binding moiety of a member of the cadherin family (e.g., cadherin E, cadherin P, cadherin VE (CD144), desmocollin 2, desmoglein 2, etc.). In some embodiments, the other binding moiety is a binding moiety of a member of the adressin family, particularly vascular addressins (e.g., PNAd, Mad, GlyCAM-1, CD34, MAdCAM-1, etc.). In certain embodiments, the polymeric drug delivery system has conjugated to it an Fc fragment and a binding moiety of a member of the cadherin family (e.g., PNAd, Mad, GlyCAM-1, CD34, MAdCAM-1, etc.). In some embodiments, the other binding moiety is a binding moiety of other adhesion molecules. In certain embodiments, the polymeric drug delivery system has conjugated to it an Fc fragment and a binding moiety of other adhesion molecules.

The nanoparticulate formulation targets receptors capable of transport across tissue, such receptors mediating transcytosis in order to cross the intestinal epithelium. The NPs can be targeted to any receptor in the intestine that participates in transport, including but not limited to the neonatal IgG Fc receptor (FcRn), Fc epsilon RH (CD23, or “low affinity” receptor for IgE), and the IgA receptor. In addition, receptors such as CD23 are upregulated during inflammation in the intestine and could be targeted for the treatment of inflammatory bowel disease with an oral formulation using NPs.

The FcRn targeting moieties can be covalently bound to the Fc-targeted nanoparticle or non-covalently bound to the R-targeted nanoparticle. Preferably the FcRn targeting moieties are covalently bound to the R-targeted nanoparticle. The FcRn targeting moieties can be on any surface of the R-targeted nanoparticle so long as they are capable of binding FcRn and facilitating transport across the intestinal epithelium. The FcRn targeting moieties are preferably bound to the outer surface of the particle core, including the outer surface of a polymeric particle core, the outer surface of a lipid particle core, or the outer surface of an inorganic particle core.

In some embodiments the FcRn targeting moiety is modified to be covalently bound to the particle core. The FcRn targeting moiety can be modified with a first reactive group capable of reacting with a second reactive group in the particle core to form a covalent bond. The choice of suitable reactive groups is within the capabilities of those skilled in the art. The FcRn targeting moiety can be covalently bound to a polymer in a polymeric particle or to a polymer in a polymer-coated hybrid particle. The FcRn targeting moiety can be covalently bound to a lipid in a lipid particle or to a lipid on the surface of a lipid stabilized particle. The FcRn targeting moiety can be covalently bound to a metal in an inorganic particle or to a capping ligand on the surface of an inorganic particle.

The FcRn targeting moiety can be bound at any density on the surface of an Fc-targeted nanoparticle that provides targeting to and transport across the intestinal epithelium. Preferably, the FcRn targeting moiety is covalently bound at a density of at least 10 moieties per square micron, especially for ligands such as antibodies. In some embodiments, especially those using low molecular weight moieties, the moieties can be bound in a ratio of greater than 1 mg of targeting moiety to 10,000 mg of particle, greater than 1 mg of targeting moiety to 1,000 mg of particle, greater than 1 mg of targeting moiety to 500 mg of particle, greater than 1 mg of targeting moiety to 400 mg of particle, greater than 1 mg of targeting moiety to 300 mg of particle, greater than 1 mg of targeting moiety to 200 mg of particle, or greater than 1 mg of targeting moiety to 100 mg of particle.

C. Therapeutic, Prophylactic, and Diagnostic Agents

Nanoparticles (NPs) can be used to treat many diseases, including cancer, cardiovascular disease, and diabetes. Therapeutic, prophylactic and diagnostic agents can be proteins, peptides, carbohydrates, lipids, small molecules, nucleic acid (DNA, RNA, siRNA, mRNA, microRNA, ribozymes, triplex forming oligonucleotides), or combinations thereof. The NPs, not just the drug, enter systemic circulation after oral administration. For example, NPs targeted to the FcRn were able to enter circulation and reach several organs, including the lungs, liver, spleen, heart, and kidneys.

The NPs can be targeted to the receptor for mucosal vaccination. Targeted NPs delivering both antigen and adjuvant with the formulation, the NPs could be used to elicit an immunological response for oral or intranasal immunization.

Because the NPs showed distribution to the kidneys after oral administration, therapeutic agents effective for treating hypertension, heart failure, or another condition associated with renal activity can be treated using this drug delivery system. Treatable conditions can also include renal conditions such as kidney stones, kidney infections, and kidney cancers. Examples of suitable functional classes of drugs include diuretics, aldosterone II receptor antagonists, vasodilators, calcium-channel blockers, renin inhibitors, nerve inhibitors, local anesthetics, angiotensin II receptor blockers, ACE inhibitors, anti-inflammatories, antibiotics, endotheiin-receptor antagonists, receptor agonists, among others. Examples of suitable drugs and drug types include bumetanide, furosemide, natriuretic peptides (e.g., atrial natriuretic peptides, brain natriuretic peptides, and C-type natriuretic peptides), spironolactone, eplerenone, isosorbide, isosorbide dinitrate, isosorbide-5-mononitrate, apresoline, aliskiren (e.g., TEKTURNA aliskiren), chlorothiazide (e.g., DIURIL chlorothiazide), indapamide, lidocaine, procaine, hypertonic solutions (e.g., high-concentration NaCl), amlodipine (e.g., NORVASC amlodipine), losartan (e.g., HYZAAR losartan potassium and hydrochlorothiazide), bosentan, clonidine (e.g., CATAPRES clonidine), enalapril, lisinopril, captopril, carvedilol, metoprolol, bisoprolol, nitric oxide (NO), compounds that are capable of generating NO in situ (e.g., glyceryl trinitrate, isoamyl nitrite, sodium nitroprusside, molsidomine, S-nitrosoglutathione, and other suitable NO-donor compounds), antibodies, peptides, siRNAs, and polynucleotides that encode polypeptides that affect renal activity, among others.

Because of NP distribution to the lungs after oral administration, therapeutic agents such as proteins, peptide, bronchodilators, corticosteroids, elastase inhibitors, analgesics, antifungals, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, fungal infection therapies, respiratory illness therapies associated with acquired immune deficiency syndrome, an oncology drug, an anti-emetic, an analgesic, and a cardiovascular agent can be delivered using this system.

For therapy of lung cancer or bronchial dysplasia, exemplary drugs include Paclitaxel, Gefitinib, Erlotinib, Etoposide, Carboplatin, Docetaxel, Vinorelbine tartrate, Cisplatin, Doxorubicin, Ifosfamide, Vincristine sulfate, Gemcitabine hydrochloride, Lomustine (CCNU), Cyclophosphamide, Methotrexate, Topotecan hydrochlorid, irinotecan, 5-fluorouracil, Zileuton, Celecoxib, and their derivatives, wherein the derivatives of the drugs are preferably fatty acid derivatives, in particular palmitic acid derivatives, such as Paclitaxel palmitate may be.

In the diagnosis and/or therapy of lung cancer or bronchial dysplasia, the active agent is a radiopharmaceutical such as Calcium-47, Carbon-11, Carbon-14, Chromium-51, Cobalt-57, Cobalt-58, Erbium-169, Fluorine-18, Gallium-67, Gallium-68, Hydrogen-3, Indium-111, Iodine-123, Iodine-131, Iron-59, Krypton-81 m, Nitrogen-13, Oxygen-15, Phosphorus-32, Samarium-153, Selenium-75, Sodium-22, Sodium-24, Strontium-89, Technetium-99m, Thallium-201, Xenon-133, Yttrium-90, and substances comprising at least one of the radionuclides.

For the use in diagnosis or imaging methods, particularly by PET and/or CT, it is preferred if the radiopharmaceutical is Technetium-99m (e.g. in Technetium-99m scintigraphy or CT) or Fluorine 18-FDG (e.g. in Fluorine 18-FDG PET). In the diagnosis of lung cancer or bronchial dysplasia, the active agent is a contrasting agent such as iodine-, gadolinium-, magnetite-, or fluorine-containing contrasting agents, wherein the contrasting agent is preferably an iodine-containing agent, in particular, iopromide, ioxitalamate, ioxaglate, iohexol, iopamidol, iotralon, or metrizamide.

Anti-cancer active agents can be alkylating agents, antimetabolites, natural products, hormones and antagonists, and miscellaneous agents, such as radiosensitizers. Examples of alkylating agents include: alkylating agents having the bis-(2 chloroethyl)-amine group such as chlormethine, chlorambucile, melphalan, uramustine, mannomustine, extramustinephoshate, mechlore-thaminoxide, cyclophosphamide, if osfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carmustine, lomustine, semustine, or; streptozotocine; and alkylating agents of the mitobronitole, dacarbazine and procarbazine type. Examples of anti-metabolites include: folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine. Examples of natural products include: vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifiers, such as alpha-interferon; camptothecin; taxol; and retinoids, such as retinoic acid. Examples of hormones and antagonists include: adrenocorticosteroids, such as prednisone; progestins, such as hydroxyprogesterone caproate, medroxyprogesterone acetate, and megestrol acetate; estrogens, such as diethylstilbestrol and ethinyl estradiol; anti-estrogens, such as tamoxifen; androgens, such as testosterone propionate and fluoxymesterone; anti-androgens, such as flutamide; and gonadotropin-releasing hormone analogs, such as leuprolide. Examples of miscellaneous agents include: radiosensitizers, such as 1,2,4-benzotriazin-3-amine 1,4-dioxide (SR 4889) and 1,2,4-benzotriazine 7-amine 1,4-dioxid; substituted ureas, such as hydroxyurea; and adrenocortical suppressants, such as mitotane and aminoglutethimide.

The anticancer agent can be an immunosuppressive drug, such as cyclosporine, azathioprine, sulfasalazine, methoxsalen, and thalidomide. Analgesic active agents, include, for example, an NSAID or a COX-2 inhibitor. Exemplary NSAIDS includenabumetone, tiaramide, proquazone, bufoxamac, flumizole, epirazole, tinoridine, timegadine, and dapsone. Suitable acidic compounds include, for example, carboxylic acids and enolic acids. Suitable carboxylic acid NSAIDs include, for example: salicylic acids and esters thereof, such as aspirin, diflunisal, benorylate, and fosfosal; acetic acids, such as phenylacetic acids, including diclofenac, alclofenac, and fenclofenac; carbo- and heterocyclic acetic acids such as etodolac, indomethacin, sulindac, tolmetin, fentiazac, and tilomisole; propionic acids, such as carprofen, fenbulen, flurbiprofen, ketoprofen, oxaprozin, suprofen, tiaprofenic acid, ibuprofen, naproxen, fenoprofen, indoprofen, and pirprofen; and fenamic acids, such as flutenamic, mefenamic, meclofenamic, and niflumic. Suitable enolic acid NSAIDs include, for example: pyrazolones such as oxyphenbutazone, phenylbutazone, apazone, and feprazone; and xicams such as piroxicam, sudoxicam, isoxicam, and tenoxicam. Exemplary COX-2 inhibitors include, but are not limited to, celecoxib (SC-58635, CELEBREX®, Pharmacia/Searle & Co.), rofecoxib (MK 966, L-74873 1 , VIOXX®, Merck & Co.), meloxicam (MOBIC®, co-marketed by Abbott Laboratories, Chicago, Ill., and Boehringer lngelheim Pharmaceuticals), valdecoxib (BEXTRA®, G. D. Searle & Co.), parecoxib (G. D. Searle & Co.), etoricoxib (MK-663; Merck), benzenesulfonamide; G. D. Searle & Co., Skokie, Ill.); piroxicam (FELDANE®; Pfizer3; diclofenac (VOLTAREN® and CATAFLAM®, Novartis);.

By conjugating IgG Fc fragments to the NP surface, the NPs could be targeted to the FcRn after oral administration. In acidic sections of the intestine, such as the duodenum and portions of the jejunum, Fc fragments conjugated to NPs [Fc-targeted NPs (NP-Fc)] will bind to FcRn at the apical surface of absorptive epithelial cells, leading to receptor-mediated transcytosis. NP-Fc could also be taken up by fluid phase pinocytosis. During intracellular trafficking, NP-Fc and FcRn in the same acidic endosome compartments will bind with high affinity. FcRn can then guide bound NP-Fc through a transcytosis pathway, avoiding lysosomal degradation. On the basolateral side, exocytosis results in exposure to a neutral pH environment in the lamina propria, causing the release of NP-Fc. NP-Fc can then diffuse through the lamina propria and enter systemic circulation.

The loading range for the agent within the particles is from about 0.01 to about 80% (agent weight/particle core weight), preferably from 0.01% to about 50% (wt/wt), more preferably from about 0.01% to about 25% (wt/wt), even more preferably from about 0.01% to about 10% (wt/wt), most preferably from about 0.1% to about 5% (wt/wt). For small molecules, the percent loading is typically from about 0.01% to about 20% (wt/wt), although higher loadings may be achieved for cores containing agent alone without polymer, lipid, etc. and/or for hydrophobic drugs and/or insoluble metals. For large biomolecules, such as proteins and nucleic acids, typical loadings are from about 0.01% to about 5% (wt/wt), preferably from about 0.01% to about 2.5% (wt/wt), more preferably from about 0.01% to about 1% (wt/wt). The loading can be calculated relative to the mass of the polymer, lipid, or inorganic particles.

The drug delivery system targets receptors capable of transport through tissue, for example, receptors that elicit transcytosis in order to cross the intestinal epithelium. The NPs can be targeted to any receptor in Table 1 for selective delivery into the listed tissue. For example, the NPs can be targeted to receptors in the intestine that participates in transcytosis, including but not limited to the neonatal IgG Fc receptor (FcRn), Fc epsilon RII (CD23, or “low affinity” receptor for IgE), and the IgA receptor. In addition, receptors such as CD23 are upregulated during inflammation in the intestine and can be targeted for the treatment of inflammatory bowel disease with an oral formulation using NPs.

The NP formulation also relates to the use of NPs targeted to the receptor for mucosal vaccination. Targeted NPs delivering both antigen and adjuvant with the formulation, the NPs can be used to elicit an immunological response, for example by subcutaneous, oral or topical (intranasal) immunization.

The NP formulation also relates to the ability of the NPs, not just the drug, to enter systemic circulation after oral administration. For example, NPs targeted to the FcRn were able to enter circulation and reach several organs, including the lungs, liver, spleen, heart, and kidneys. This offers the opportunity to deliver drugs to each of these organs after oral administration in a controlled manner from the NPs. Because the NPs showed distribution to the kidneys after oral administration, therapeutic agents effective for treating hypertension, heart failure, or another condition associated with renal activity can be treated using this drug delivery system. Treatable conditions could also include renal conditions such as kidney stones, kidney infections, and kidney cancers. Examples of suitable functional classes of drugs include diuretics, aldosterone II receptor antagonists, vasodilators, calcium-channel blockers, renin inhibitors, nerve inhibitors, local anesthetics, angiotensin II receptor blockers, ACE inhibitors, anti-inflammatories, antibiotics, endotheiin-receptor antagonists, receptor agonists, among others. Examples of suitable drugs and drug types include bumetanide, furosemide, natriuretic peptides (e.g., atrial natriuretic peptides, brain natriuretic peptides, and C-type natriuretic peptides), spironolactone, eplerenone, isosorbide, isosorbide dinitrate, isosorbide-5-mononitrate, apresoline, aliskiren (e.g., TEKTURNA aliskiren), chlorothiazide (e.g., DIURIL chlorothiazide), indapamide, lidocaine, procaine, hypertonic solutions (e.g., high-concentration NaCl), amlodipine (e.g., NORVASC amlodipine), losartan (e.g., HYZAAR losartan potassium and hydrochlorothiazide), bosentan, clonidine (e.g., CATAPRES clonidine), enalapril, lisinopril, captopril, carvedilol, metoprolol, bisoprolol, nitric oxide (NO), compounds that are capable of generating NO in situ (e.g., glyceryl trinitrate, isoamyl nitrite, sodium nitroprusside, molsidomine, S-nitrosoglutathione, and other suitable NO-donor compounds), antibodies, peptides, siRNAs, and polynucleotides that encode polypeptides that affect renal activity, among others.

Because of NP distribution to the lungs after oral administration, therapeutic agents such as proteins, peptide, bronchodilators, corticosteroids, elastase inhibitors, analgesics, antifungals, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, fungal infection therapies, respiratory illness therapies associated with acquired immune deficiency syndrome, an oncology drug, an anti-emetic, an analgesic, and a cardiovascular agent could be delivered using this system.

For therapy of lung cancer or bronchial dysplasia, the drug can be Paclitaxel, Gefitinib, Erlotinib, Etoposide, Carboplatin, Docetaxel, Vinorelbine tartrate, Cisplatin, Doxorubicin, Ifosfamide, Vincristine sulfate, Gemcitabine hydrochloride, Lomustine (CCNU), Cyclophosphamide, Methotrexate, Topotecan hydrochlorid, irinotecan, 5-fluorouracil, Zileuton, Celecoxib, and their derivatives, wherein the derivatives of the drugs are preferably fatty acid derivatives, in particular palmitic acid derivatives, such as Paclitaxel palmitate.

For diagnosis and/or therapy of lung cancer or bronchial dysplasia, the active agent is a radiopharmaceutical selected from the group consisting of Calcium-47, Carbon-11, Carbon-14, Chromium-51, Cobalt-57, Cobalt-58, Erbium-169, Fluorine-18, Gallium-67, Gallium-68, Hydrogen-3, Indium-111, Iodine-123, Iodine-131, Iron-59, Krypton-81 m, Nitrogen-13, Oxygen-15, Phosphorus-32, Samarium-153, Selenium-75, Sodium-22, Sodium-24, Strontium-89, Technetium-99m, Thallium-201, Xenon-133, Yttrium-90, and substances comprising at least one of the radionuclides. For the use in diagnosis or imaging methods, particularly by PET and/or CT, it is preferred if the radiopharmaceutical is Technetium-99m (e.g. in Technetium-99m scintigraphy or CT) or Fluorine 18-FDG (e.g. in Fluorine 18-FDG PET). In a further preferred embodiment, the active agent is a contrasting agent selected from the group consisting of iodine-, gadolinium-, magnetite-, or fluorine-containing contrasting agents, wherein the contrasting agent is preferably selected from the group of the iodine-containing agents, in particular from the group consisting of iopromide, ioxitalamate, ioxaglate, iohexol, iopamidol, iotralon, and metrizamide.

Anti-cancer active agents are preferably selected from alkylating agents, antimetabolites, natural products, hormones and antagonists, and miscellaneous agents, such as radiosensitizers. Examples of alkylating agents include: alkylating agents having the bis-(2 chloroethyl)-amine group such as chlormethine, chlorambucile, melphalan, uramustine, mannomustine, extramustinephoshate, mechlore-thaminoxide, cyclophosphamide, if osfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carmustine, lomustine, semustine, or; streptozotocine; and alkylating agents of the mitobronitole, dacarbazine and procarbazine type.

Examples of anti-metabolites include folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine.

Examples of natural products include vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifers, such as alpha-interferon; camptothecin; taxol; and retinoids, such as retinoic acid. Examples of hormones and antagonists include adrenocorticosteroids, such as prednisone; progestins, for example, hydroxyprogesterone caproate, medroxyprogesterone acetate, and megestrol acetate; estrogens, such as diethylstilbestrol and ethinyl estradiol; anti-estrogens, such as tamoxifen; androgens, such as testosterone propionate and fluoxymesterone; anti-androgens, such as flutamide; and gonadotropin-releasing hormone analogs, such as leuprolide. Examples of miscellaneous agents include radiosensitizers, such as 1,2,4-benzotriazin-3-amine 1,4-dioxide (SR 4889) and 1,2,4-benzotriazine 7-amine 1,4-dioxide (WIN 59075); platinum coordination complexes such as cisplatin and carboplatin; anthracenediones, such as mitoxantrone; substituted ureas, such as hydroxyurea; and adrenocortical suppressants, such as mitotane and aminoglutethimide. In addition, the anticancer agent can be an immunosuppressive drug, such as cyclosporine, azathioprine, sulfasalazine, methoxsalen, and thalidomide.

Many different active agents can be administered using this system. In a particularly preferred embodiment, the delivery system is used to deliver a peptide which is not normally orally bioavailable. An example is insulin.

Insulin or insulin analogs may be used in this formulation. Preferably, the insulin is recombinant human insulin. Recombinant human insulin is available from a number of sources. The dosages of the insulin depend on its bioavailability and the patient to be treated. Insulin is generally included in a dosage range of 1.5-200 IU, depending on the level of insulin resistance of the individual. Typically, insulin is provided in 100 IU vials, though other presentations of 200, 400 or 500 U/ml are described herein. In the most preferred embodiment the injectable formulation is a volume of 1 ml containing 100 U of insulin. Additional embodiments include higher concentration insulin formulations, the most preferred being U-400.

There are several differing types of commercial insulin available for diabetes patients. These types of insulins vary according to (1) how long they take to reach the bloodstream and start reducing blood glucose levels; (2) how long the insulin operates at maximum strength; and (3) how long the insulin continues to have an effect on blood sugar.

Fast acting insulins are intended to respond to the glucose derived from ingestion of carbohydrates during a meal. Fast acting insulins start to work within one to 20 minutes, peaking about one hour later and lasting from three to five hours. Fast acting insulin takes about two hours to fully absorb into the systemic circulation. Fast acting insulins include regular recombinant human insulin (such as HUMULIN®, marketed by Eli Lilly, and NOVALIN®, marketed by Novo Nordisk A/S) which are administered in an isotonic solution at pH 7. Bovine and porcine insulins, which differ in several amino acids to human insulin, but are bioactive in humans, are also fast acting insulins.

More concentrated forms of insulin are provided for insulin resistant individuals. The commercially available formulation Humulin R U-500 has a very long duration of action and is suitable for basal use only due to its slow release profiles.

Some diabetes patients use rapid-acting insulin at mealtimes, and long-acting insulin for ‘background’ continuous insulin. This group includes insulins that have been modified or have altered locations of amino acids in order to enhance their rate of absorption.

At present there are three types of rapid-acting commercial insulin analogs available: insulin lispro (Lysine-Proline insulin, sold by Eli Lilly as HUMALOG®), insulin glulisine (sold by Sanofi-Aventis as APIDRA®) and insulin aspart (sold by Novo Nordisk as NOVOLOG®).

Intermediate-acting insulin has a longer lifespan than short-acting insulin but it is slower to start working and takes longer to reach its maximum strength. Intermediate-acting insulin usually starts working within 2-4 hours after injection, peaks somewhere between 4-14 hours and remains effective up to 24 hours. Types of intermediate-acting insulin include NPH (Neutral Protamine Hagedorn) and LENTE insulin. NPH insulin contains protamine which slows down the speed of absorption so that the insulin takes longer to reach the bloodstream but has a longer peak and lifespan. Intermediate acting insulins may be combined with rapid acting insulins at neutral pH, to reduce the total number of injections per day.

Blends of immediate acting insulin and intermediate acting insulin: Blends of rapid acting insulin and NPH insulin are commercially available to fulfill the need for prandial and basal use in a single injection. These insulin blends may be regular recombinant insulin based (HUMULIN® 70/30 (70% human insulin isophane and 30% human insulin, Eli Lilly) or analog based, such HUMALOG®Mix75/25 (75% insulin lispro protamine suspension and 25% insulin lispro solution) (Eli Lilly) and are 100 U-ml. These blends use a protamine insulin suspension (HUMULIN® or HUMALOG® based) to extend the duration of action insulin action with HUMULIN®R (regular human insulin) or HUMALOG®R to cover the prandial needs.

Examples of long acting insulins are insulin glargine (marketed under the tradename LANTUS®, Sanofi Aventis) and insulin detemir (LEVEMIR®, Novo Nordisk A/S). The extended duration of action of LANTUS® is normally induced by the pH elevation from 4 to 7 post subcutaneous injection. This changes the solubility of the insulin glargine, creating a microprecipitate. This microprecipitate slowly dissolves in the subcutaneous tissue, sustaining its glucose lowering effect for up to 24 hours. It differs from human insulin by having a glycine instead of asparagine at position 21 and two arginines added to the carboxy-terminus of the beta-chain.

### III. Methods of Making R-Targeted Nanoparticles

Methods of making nanoparticles include precipitation, stretching, molding (PRINT), grinding, litography, microfluidic, or other methods to prepare nanoparticles. The nanoparticle's shape may be spherical, rod-like, cube-like, tripod-like, tetrapod-like, elipsoid-like, disk-like, or worm-like. The nanoparticles may be porous, solid, high density, low density properties.

Polymer-drug conjugates can be prepared using synthetic methods known in the art. Representative methodologies for the preparation of polymer-drug conjugates are discussed below. The appropriate route for synthesis of a given polymer-drug conjugate can be determined in view of a number of factors, such as the structure of the polymer-drug conjugate, the composition of the polymer segments which make up the polymer-drug conjugate, the identity of the one or more drugs attached to the polymer-drug conjugate, as well as the structure of the conjugate and its components as it relates to compatibility of functional groups, protecting group strategies, and the presence of labile bonds.

In addition to the synthetic methodologies discussed below, alternative reactions and strategies useful for the preparation of the polymer-drug conjugates disclosed herein are known in the art. See, for example, March, “Advanced Organic Chemistry,” 5^{th} Edition, 2001, Wiley-Interscience Publication, New York).

A. Particle Core

i. Polymeric Particle Core

Methods of making polymeric particles are known in the art. Polymeric particles useful as a polymeric particle core can be prepared using any suitable method known in the art. Common techniques include, but are not limited to, spray drying, phase separation encapsulation (spontaneous emulsion encapsulation, solvent evaporation encapsulation, and solvent removal encapsulation), coacervation, and phase inversion nanoencapsulation (PIN). A brief summary of these methods is presented below.

1. Spray Drying

Methods for forming polymeric particles using spray drying techniques are described in U.S. Pat. No. 6,620,617, to Mathiowitz et al. In this method, the polymer is dissolved in an organic solvent such as methylene chloride or in water. A known amount of one or more active agents to be incorporated in the particles is suspended (in the case of an insoluble active agent) or co dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Nanoparticles/nanospheres ranging between 0.1 10 microns can be obtained using this method.

2. Phase Separation Encapsulation

In phase separation nanoencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

a. Spontaneous Emulsion Nanoencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

b. Solvent Evaporation Nanoencapsulation

Methods for forming nanoparticles using solvent evaporation techniques are described in E. Mathiowitz et al., J. Scanning Microscopy, 4:329 (1990); L. R. Beck et al., Fertil. Steril., 31:545 (1979); L. R. Beck et al, Am J Obstet Gynecol., 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid nanoparticles/nanoparticles. This method is useful for relatively stable polymers like polyesters and polystyrene.

c. Solvent Removal Nanoencapsulation

The solvent removal nanoencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Nanoparticles that range between 1-300 microns can be obtained by this procedure. Substances which can be incorporated in the nanoparticles include pharmaceuticals, pesticides, nutrients, imaging agents, and metal compounds.

3. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric nanoparticles, wherein the polymer is either coated with the particles or the particles are dispersed in the polymer. See, U.S. Pat. No. 6,143,211 to Mathiowitz, et al. The method can be used to produce monodisperse populations of nanoparticles and nanoparticles in a wide range of sizes, including, for example, about 100 nanometers to about 10 microns. Advantageously, an emulsion need not be formed prior to precipitation. The process can be used to form nanoparticles from thermoplastic polymers.

ii. Lipid Particle Core

Methods of making lipid particles are known in the art. Lipid particles useful as a lipid particle core can be lipid micelles, liposomes, or solid lipid particles prepared using any suitable method known in the art. Common techniques for creating lipid particles encapsulating an active agent include, but are not limited to, high pressure homogenization techniques, supercritical fluid methods, emulsion methods, solvent diffusion methods, and spray drying. A brief summary of these methods is presented below.

1. High Pressure Homogenization (HPH) Methods

High pressure homogenization is a reliable and powerful technique, which is used for the production of smaller lipid particles with narrow size distributions, including lipid micelles, liposomes, and solid lipid particles. High pressure homogenizers push a liquid with high pressure (100-2000 bar) through a narrow gap (in the range of a few microns). The fluid can contain lipids that are liquid at room temperature or a melt of lipids that are solid at room temperature. The fluid accelerates on a very short distance to very high velocity (over 1000 Km/h). This creates high shear stress and cavitation forces that disrupt the particles, generally down to the submicron range. Generally 5-10% lipid content is used but up to 40% lipid content has also been investigated.

Two approaches of HPH are hot homogenization and cold homogenization, work on the same concept of mixing the drug in bulk of lipid solution or melt.

a. Hot Homogenization:

Hot homogenization is carried out at temperatures above the melting point of the lipid and can therefore be regarded as the homogenization of an emulsion. A pre-emulsion of the drug loaded lipid melt and the aqueous emulsifier phase is obtained by a high-shear mixing. HPH of the pre-emulsion is carried out at temperatures above the melting point of the lipid. A number of parameters, including the temperature, pressure, and number of cycles, can be adjusted to produce lipid particles with the desired size. In general, higher temperatures result in lower particle sizes due to the decreased viscosity of the inner phase. However, high temperatures increase the degradation rate of the drug and the carrier. Increasing the homogenization pressure or the number of cycles often results in an increase of the particle size due to high kinetic energy of the particles.

b. Cold Homogenization

Cold homogenization has been developed as an alternative to hot homogenization. Cold homogenization does not suffer from problems such as temperature-induced drug degradation or drug distribution into the aqueous phase during homogenization. The cold homogenization is particularly useful for solid lipid particles, but can be applied with slight modifications to produce liposomes and lipid micelles. In this technique the drug containing lipid melt is cooled, the solid lipid ground to lipid nanoparticles and these lipid nanoparticles are dispersed in a cold surfactant solution yielding a pre-suspension. The pre-suspension is homogenized at or below room temperature, where the gravitation force is strong enough to break the lipid nanoparticles directly to solid lipid nanoparticles.

2. Ultrasonication/High Speed Homogenization Methods

Lipid particles, including lipid micelles, liposomes, and solid lipid particles, can be prepared by ultrasonication/high speed homogenization. The combination of both ultrasonication and high speed homogenization is particularly useful for the production of smaller lipid particles. Liposomes are formed in the size range from 10 nm to 200 nm, preferably 50 nm to 100 nm, by this process.

3. Solvent Evaporation Methods

Lipid particles can be prepared by solvent evaporation approaches. The lipophilic material is dissolved in a water-immiscible organic solvent (e.g. cyclohexane) that is emulsified in an aqueous phase. Upon evaporation of the solvent, nanoparticles dispersion is formed by precipitation of the lipid in the aqueous medium. Parameters such as temperature, pressure, choices of solvents can be used to control particle size and distribution. Solvent evaporation rate can be adjusted through increased/reduced pressure or increased/reduced temperature.

4. Solvent Emulsification-Diffusion Methods

Lipid particles can be prepared by solvent emulsification-diffusion methods. The lipid is first dissolved in an organic phase, such as ethanol and acetone. An acidic aqueous phase is used to adjust the zeta potential to induce lipid coacervation. The continuous flow mode allows the continuous diffusion of water and alcohol, reducing lipid solubility, which causes thermodynamic instability and generates liposomes

5. Supercritical Fluid Methods

Lipid particles, including liposomes and solid lipid particles, can be prepared from supercritical fluid methods. Supercritical fluid approaches have the advantage of replacing or reducing the amount of the organic solvents used in other preparation methods. The lipids, active agents to be encapsulated, and excipients can be solvated at high pressure in a supercritical solvent. The supercritical solvent is most commonly CO₂, although other supercritical solvents are known in the art. To increase solubility of the lipid, a small amount of co-solvent can be used. Ethanol is a common co-solvent, although other small organic solvents that are generally regarded as safe for formulations can be used. The lipid particles, lipid micelles, liposomes, or solid lipid particles can be obtained by expansion of the supercritical solution or by injection into a non-solvent aqueous phase. The particle formation and size distribution can be controlled by adjusting the supercritical solvent, co-solvent, non-solvent, temperatures, pressures, etc.

6. Microemulsion Based Methods

Microemulsion based methods for making lipid particles are known in the art. These methods are based upon the dilution of a multiphase, usually two-phase, system. Emulsion methods for the production of lipid particles generally involve the formation of a water-in-oil emulsion through the addition of a small amount of aqueous media to a larger volume of immiscible organic solution containing the lipid. The mixture is agitated to disperse the aqueous media as tiny droplets throughout the organic solvent and the lipid aligns itself into a monolayer at the boundary between the organic and aqueous phases. The size of the droplets is controlled by pressure, temperature, the agitation applied and the amount of lipid present.

The water-in-oil emulsion can be transformed into a liposomal suspension through the formation of a double emulsion. In a double emulsion, the organic solution containing the water droplets is added to a large volume of aqueous media and agitated, producing a water-in-oil-in-water emulsion. The size and type of lipid particle formed can be controlled by the choice of and amount of lipid, temperature, pressure, co-surfactants, solvents, etc.

7. Spray Drying Methods

Spray drying methods similar to those described above for making polymeric particle can be employed to create solid lipid particles. This works best for lipid with a melting point above 70° C.

iii. Inorganic Particle Core

Methods of making inorganic particles are known in the art. Inorganic particles useful as an inorganic particle core can be metal particles, semiconductor particles, or metal oxide particles prepared using any suitable method known in the art. Suitable methods of making inorganic particles can include those described in Altavilla, C., and Ciliberto, E., eds. Inorganic Nanoparticles: Synthesis, Applications, and Perspectives. CRC Press, 2010; and Rao et al., Dalton Trans., 41:5089-5120 (2012). Common techniques for created inorganic particles include, but are not limited to physical preparation methods, gas-phase and solution-phase chemical preparation methods, and thermolysis methods. A brief summary of these methods is presented below. In some embodiments the inorganic particle cores encapsulate an active agent. In some embodiments, for example in imaging applications, the inorganic particle core is the active agent.

1. Physical Preparation Methods

Inorganic particles can be produced by physical preparation methods. Physical preparation methods generally involve the formation of a metal or a metal oxide vapor, typically in a low-pressure or vacuum environment, and coagulation and condensation of particles onto a substrate. The process typically involves use of an inert carrier gas such as He, Ne, or Ar. The heat sources can include electrical sources such as wires or filaments, lasers, or plasma arcs. This is particularly useful for producing elemental particles such as Ag, Fe, Ni, or Ga. Other inorganic particles that can be produced by physical methods can include TiO₂, SiO₂, and PbS particles.

Inorganic particles are often prepared from metal precursors. Metal precursors can include bulk metals, metal halides, metal alkoxides, metal salts,

2. Chemical Preparation Methods

Inorganic particles can be prepared by a variety of chemical preparation methods known in the art. Chemical preparation methods can be conducted in the gas phase or in solution phase. Typically, the monomers used to form the particles are produced by a chemical reaction starting from highly reactive precursors. The reaction may occur spontaneously or may be driven by heat, light, or catalyst.

The inorganic particle can be prepared by reduction of a halide precursor. The basic approach is to have some compound, typically a halide, containing a metal atom, as well as a reducing agent which removes the other parts of the compound. The halide can be F, Cl, Br, or I. An example includes making Mo particles by the reduction of MoCl₃, i.e with NaB(CH₂)₃H.

The inorganic particles can be prepared by the oxidation of a suitable precursor. For example, TiO₂ particles can be prepared by oxidation of the tetrachloride precursor TiCl₄. For example, this can be done with an oxygen plasma.

3. Thermolysis

Nanoparticles can also be made by decomposing solid or liquid precursors, often at high temperatures. For example, Li particles can be made by decomposing the solid lithium azide, LiN₃. Al nanoparticles can be made by decomposing (CH₃)₂(CH₂)NAlH₃ in toluene at 105° C. With a Ti catalyst, this leads to the production of 80 nm particles.

4. Stabilization of Inorganic Particles

In some embodiments it will be necessary to stabilize the inorganic particles, for example to prevent degradation or aggregation. Suitable capping ligands for stabilizing inorganic particles are known in the art.

5. Microfluidics

Methods of making nanoparticles using microfluidics are known in the art. Suitable methods include those described in U.S. Patent Application Publication No. 2010/0022680 A1 by Karnik et al. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the nanoparticles having the desired size and density of moieties on the surface. By varying the pressure and flow rate in the inlet channels and the nature and composition of the fluid sources nanoparticles can be produced having reproducible size and structure.

B. Receptor Targeting Moieties

Targeting moieties are adsorbed, absorbed, conjugated, complexed, bound, or assembled to the nanoparticle materials prior to assembly or after assembly of the nanoparticles. In some embodiments the transcytosis receptor targeting moieties are non-covalently attached to the surface of the R-targeted nanoparticle. For example, the transcytosis receptor targeting moieties can be bound by electrostatic or Van der Walls interactions. In these embodiments the FcRn targeting moieties can be attached by placing the particle core in a solution containing the transcytosis receptor targeting moieties. By controlling the concentration of the targeting moieties and the particles in the solution and by controlling the electrostatic properties of the particle surface, the density of transcytosis receptor targeting moieties can be controlled. In another embodiment, Fc can be absorbed to NP surfaces.

In some embodiments the transcytosis receptor targeting moieties are covalently bound to the particle core after formation of the particle. In some embodiments the transcytosis receptor targeting moieties are modified to have a first reactive group. In preferred embodiments the transcytosis receptor targeting moiety is modified to include a thiol group. The transcytosis receptor targeting moiety can be modified to have a thiol group using 2-iminothiolane. The first reactive group can be reacted with a second reactive group on the particle core to form a covalent bond. In some embodiments the second reactive group is a maleimide group. The maleimide group can be in a polymer, a lipid, a capping ligand for an inorganic particle, or any combination thereof.

In some embodiments the transcytosis receptor targeting moieties are covalently bound to the materials used to form the polymer core prior to forming the particles. In some embodiments the transcytosis receptor targeting moieties are modified to have a first reactive group. In preferred embodiments the transcytosis receptor targeting moiety is modified to include a thiol group. The transcytosis receptor targeting moiety can be modified to have a thiol group using 2-iminothiolane. The first reactive group can be reacted with a second reactive group on a polymer, lipid, or capping ligand that will be used to form the particle core to form a covalent bond. In some embodiments the second reactive group is a maleimide group. The polymer, lipid, or capping ligand having the transcytosis receptor targeting moiety covalently bound can then be used to form the particles as described already above.

Particles may have more than one type of receptor on their surface. For example, one type of receptor may be utilized for initial uptake, then a second type of receptor used to direct transport through the cells by transcytosis. For example, the nanoparticles may include a FcRN receptor to target delivery of NPs to the brain, where a transferrin receptor is exposed to facilitate transfer into brain tissue. This would avoid the problem with the transferrin receptor binding to albumin during systemic administration, rendering the NPs incapable of effective transport into the brain. This may represent a means to pass through the blood brain barrier.

### IV. Compositions and Formulations of R-Targeted Nanoparticles

The formulations described herein contain an effective amount of R-targeted nanoparticles in a pharmaceutical carrier appropriate for administration to an individual in need thereof. The formulations can be administered enterally, parenterally (e.g., by injection or infusion), topically (e.g., to the eye or a mucosal surface such as the oral cavity, intranasal, intravaginal or intrarectally), or via pulmonary administration. In preferred embodiments the formulations are enteral formulations. Exemplary routes of enteral administration include, but are not limited to, sublingual, buccal, and oral. Suitable dosage forms for enteral administration include, but are not limited to, tablets, capsules, caplets, solutions, suspensions, syrups, powders, or thin films.

A. Enteral Formulations

The R-targeted nanoparticles can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition which may include plasticizers, pigments, colorants, stabilizing agents, and glidants.

Formulations can be prepared using one or more pharmaceutically acceptable excipients, including diluents, preservatives, binders, lubricants, disintegrators, swelling agents, fillers, stabilizers, and combinations thereof.

Delayed release dosage formulations can be prepared as described in standard references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The R-targeted nanoparticle may be coated, for example to delay release once the particles have passed through the acidic environment of the stomach. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers and/or pH dependent polymers, with or without water insoluble/water soluble non polymeric excipient, to produce the desired release profile. The coating is either performed on dosage form (matrix or simple) which includes, but not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The enteral formulations generally contain a monodisperse plurality of the R-targeted nanoparticles. Preferably, the method used to form the R-targeted nanoparticles produces a monodisperse distribution of particles; however, methods producing polydisperse particle distributions can be used. If the method does not produce particles having a monodisperse size distribution, the particles are separated following particle formation to produce a plurality of particles having the desired size range and distribution.

Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Glidants are recommended to reduce sticking effects during film formation and drying, and will generally represent approximately 25 wt. % to 100 wt. % of the polymer weight in the coating solution.

A number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, or plasticizers. An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

V. Methods of using R-Targeted Nanoparticles and Compositions and Formulations Thereof

Nanoparticles (NPs) can be used to treat many diseases, including cancer, cardiovascular disease, and diabetes. Many NP-based therapeutics are now entering clinical trials or have been approved for use, including targeted polymeric nanoparticles. However, the impact of NPs in the clinic may be limited to a narrow set of indications because NP administration is currently restricted to parenteral methods. Many diseases that could benefit from NP-based therapeutics require frequent administration. Alternate routes of administration, particularly oral, are preferred because of the convenience and compliance by patients. Intestinal absorption of NPs is highly inefficient because the physicochemical parameters of NPs prevent their transport across cellular barriers such as the intestinal epithelium. To improve the absorption efficiency of NPs and to make the oral administration of NPs practical in the clinic, new strategies are necessary to overcome the intestinal epithelial barrier.

The nanoparticulate formulation is based on the discovery that NPs targeted to receptors in the intestine capable of trancytosis enable NPs to cross the intestinal epithelium and enter systemic circulation after oral administration. IgG Fc molecules conjugated to the surface of polymeric NPs target the NPs to the neonatal Fc receptor (FcRn). FcRn mediates IgG transport across polarized epithelial barriers. It was discovered as the receptor in the neonatal intestine that transports IgG in breast milk from mother to offspring. However, FcRn is expressed into adulthood at levels similar to fetal expression in the apical region of epithelial cells in the small intestine and diffusely throughout the colon. FcRn is also expressed in the vascular endothelium, blood-brain barrier, kidneys, liver, lungs, and throughout the hematopoietic system. FcRn interacts with the Fc portion of IgG in a pH-dependent manner, binding with high affinity in acidic (pH<6.5) but not physiological environments (pH˜7.4). By conjugating IgG Fc fragments to the NP surface, the NPs could be targeted to the FcRn after oral administration. In acidic sections of the intestine, such as the duodenum and portions of the jejunum, Fc fragments conjugated to NPs [Fc-targeted NPs (NP-Fc)] will bind to FcRn at the apical surface of absorptive epithelial cells, leading to receptor-mediated transport. NP-Fc could also be taken up by fluid phase pinocytosis. During intracellular trafficking, NP-Fc and FcRn in the same acidic endosome compartments will bind with high affinity. FcRn can then guide bound NP-Fc through a transport pathway, avoiding lysosomal degradation. On the basolateral side, exocytosis results in exposure to a neutral pH environment in the lamina propria, causing the release of NP-Fc. NP-Fc can then diffuse through the lamina propria and enter systemic circulation.

The NPs, not just the drug, enter systemic circulation after oral administration. For example, NPs targeted to the FcRn were able to enter circulation and reach several organs, including the lungs, liver, spleen, heart, and kidneys. This offers the opportunity to deliver drugs to each of these organs after oral administration in a controlled manner from the NPs. Because the NPs showed distribution to the kidneys after oral administration, therapeutic agents effective for treating hypertension, heart failure, or another condition associated with renal activity can be treated using this drug delivery system. Treatable conditions can also include renal conditions such as kidney stones, kidney infections, and kidney cancers.

The receptor(s) on the surface of the NPs are used to select the tissue where delivery is desired. See Table 1, above, for known receptors. Other receptors may also be utilized. For example, the receptors can be insulin receptor; insulin-like growth factor receptor; LDL receptor-related proteins 1 and 2 (LRP1 and LRP2); LDL receptor; Diptheria toxin receptor; Transferrin; CD23; Receptor for advanced glycation end products (RAGE); Scavenger receptor (SR); or ligands for FcRn.

For heart, skeletal muscle, or adipose tissue, the receptors would typically be gp60 or FcRn.

For testis, the receptor would be chorionic gonadotropin receptor, Insulin receptor and insulin-like growth factor receptor, FcRn, or Transferrin receptor.

For brain, one would select from insulin receptor; insulin-like growth factor receptor; LDL receptor-related proteins 1 and 2 (LRP1 and LRP2); LDL receptor; Diptheria toxin receptor; Transferrin; Receptor for advanced glycation end products (RAGE); Scavenger receptor (SR); or FcRn.

For intestine, one would select receptors for M cells; Terminal galactose (ricin B receptor); aminopeptidase N; pIgA receptor; or Cubulin/Megalin (vitamin B12); FcRn; or CD23 (for IgE).

For liver, pIgA or FcRn could be used.

For kidney, pIgA; Terminal galactose (ricin B receptor); FcRn; or Megalin could be used.

For placenta, aminopeptidase N; pIgA; FcRn; Transferrin; or Megalin may be used.

For the lungs, suitable receptors include FcRn; Transferrin; Terminal galactose (ricin B receptor); pIgA; CD23 (for IgE); and gp60.

For the mammary glands, preferred receptors include gp60; aminopeptidase N; pIgA; Transferrin; and FcRn.

For thyroid, one would use gp60 or megalin.

For the genitourinary system (including vagina), one would use pIgA, Transferrin, Megalin; gp340; FcRn; or lutropin receptor.

Receptors can also be used to target the biliary system, into and out of the eye through the cornea and into the bloodstream, the CNS, and skin/sebaceous glands.

Formulations are administered by injection, orally, or topically, typically to a mucosal surface (lung, nasal, oral, buccal, sublingual, vaginally, rectally) or to the eye (intraocularly or transocularly). Intranasal may be useful for delivery to the brain. The NPs can be targeted to the receptor for mucosal vaccination. Targeted NPs delivering both antigen and adjuvant with the formulation, the NPs could be used to elicit an immunological response for oral or intranasal immunization.

The formulations may be suspended in a carrier, applied as a powder, or prepared in excipients as described above, for example, an enteric capsule or tablet for initial passage through the stomach for release within the gastrointestinal tract.

In one embodiment, the nanoparticle formulation is administered systemically to heart, skeletal muscle, or adipose tissue. The receptors can be factor receptor; LDL receptor-related proteins 1 and 2 (LRP1 and LRP2); LDL receptor; Diptheria toxin receptor; Transferrin; Receptor for advanced glycation end products (RAGE); Scavenger receptor (SR); or ligands for FcRn.

In one embodiment, the nanoparticle formulation is administered to testis tissue, wherein the transcytosis receptors are chorionic gonadotropin receptor, Insulin receptor, insulin-like growth, or Transferrin receptor, optionally wherein the nanoparticles further include ligands for prostate specific membrane antigen.

In another embodiment the nanoparticle formulation is administered orally and the agent is taken up and passed through intestinal tissue, wherein the nanoparticles have on their surface receptors for FcRn, M cells; Terminal galactose (ricin B receptor); aminopeptidase N; pIgA receptor; or Cubulin/Megalin (vitamin B12). In another embodiment, the nanoparticle formulation is administered by injection or orally for delivery into and through liver tissue, wherein the receptor is CD23 (for IgE). In still another embodiment, the nanoparticle formulation is administered by injection or orally for delivery into and through kidney tissue, wherein the receptors are pIgA or Terminal galactose (ricin B receptor). In still another embodiment, the nanoparticle formulation is administered by injection or orally for delivery into and through placental tissue, wherein the receptors are aminopeptidase N or Megaline.

In another embodiment the nanoparticle formulation is administered by injection or orally for delivery into and through lung tissue, wherein the receptors are selected from the group consisting of ligands for FcRn; Transferrin; Terminal galactose (ricin B receptor); pIgA; FcRn; and gp60.

In yet another embodiment, the nanoparticle formulation is administered by injection or orally for delivery into and through mammary gland tissue, wherein the receptors are gp60; aminopeptidase N; or CD23 (for IgE).

In another embodiment, the nanoparticle formulation is administered by injection or orally for delivery into and through thyroid tissue, wherein the receptor is gp60.

In still another embodiment, the nanoparticle formulation is administered by injection or orally for delivery into and through genitourinary tract tissue, wherein the receptors are pIgA, Transferrin, Megalin; gp340; and/or lutropin receptor.

The nanoparticle formulation can be administered to and effectively pass through a biological barrier such as the intestinal barrier, the alveolar-blood barrier, the placental maternal-fetal barrier, the Blood-Brain-Barrier, or the retinal-blood barrier, into the adjacent tissue or vasculature.

The formulations can be used to direct transport from one tissue to another, or through a barrier into one tissue, or vice versa. For example:

In one embodiment, an effective amount of the nanoparticles are transported across the intestinal lumen and accumulate in the lamina propria and bloodstream or wherein an effective amount of the nanoparticles are transported from the bloodstream and lamina propria into the intestinal lumen and accumulate in the intestinal lumen.

In one embodiment, an effective amount of the nanoparticles are transported from the lung airway into lung tissue and bloodstream or wherein an effective amount of the nanoparticles are transported from the lung tissue and bloodstream into the lung airway and accumulate in the lung airway.

In one embodiment, an effective amount of the nanoparticles are transported from the bloodstream into the kidney tissue and filtrate and accumulate in the kidney tissue and filtrate or wherein an effective amount of the nanoparticles are transported from the kidney tissue and filtrate into the bloodstream.

In one embodiment, an effective amount of the nanoparticles are transported from the bloodstream into the mammary gland and accumulate in the mammary gland.

In one embodiment, an effective amount of the nanoparticles are transported from the mother to fetus' bloodstream and accumulate in the bloodstream of the fetus, or wherein an effective amount of the nanoparticles are transported from the fetus to mother's bloodstream and accumulate in the bloodstream of the mother.

In one embodiment, an effective amount of the nanoparticles are transported into the testis and accumulate in the testes or wherein an effective amount of the nanoparticles are transported out of the testis and deplete in the testes.

In one embodiment, an effective amount of the nanoparticles are transported from the genitourinary lumen into the genitourinary tissue and accumulate in the genitourinary tissue or wherein an effective amount of the nanoparticles are transported from the genitourinary tissue into the genitourinary lumen and accumulate in the genitourinary lumen.

In one embodiment, an effective amount of the nanoparticles are transported across the vaginal epithelium from the vaginal lumen into the vaginal tissue and accumulate in the vaginal tissue or wherein an effective amount of the nanoparticles are transported across the vaginal epithelium from the vaginal tissue into the vaginal lumen and accumulate in the vaginal lumen.

In one embodiment, an effective amount of the nanoparticles are transported from the bloodstream into the liver tissue and accumulate in the liver tissue or wherein an effective amount of the nanoparticles are transported from the bloodstream into the biliary system and accumulate in the biliary system.

In one embodiment, an effective amount of the nanoparticles are transported from the liver tissue into the bloodstream and accumulate in the bloodstream or wherein an effective amount of the nanoparticles are transported from the biliary system into the bloodstream and accumulate in the bloodstream.

In one embodiment, an effective amount of the nanoparticles are transported from the liver tissue into the biliary system and accumulate in the biliary system or wherein an effective amount of the nanoparticles are transported from the biliary system into the liver tissue and accumulate in the liver tissue.

In one embodiment, an effective amount of the nanoparticles are transported from the surface of the eye into the ocular tissue and accumulate in the ocular tissue or wherein an effective amount of the nanoparticles are transported from the ocular tissue onto the surface of the eye and accumulate on the eye surface.

In one embodiment, an effective amount of the nanoparticles are transported from the surface of the eye into the bloodstream and accumulate in the bloodstream or wherein an effective amount of the nanoparticles are transported from the bloodstream onto the surface of the eye and accumulate on the eye surface.

In one embodiment, an effective amount of the nanoparticles are transported from the ocular tissue into the bloodstream and accumulate in the bloodstream or wherein an effective amount of the nanoparticles are transported from the bloodstream into the ocular tissue of the eye and accumulate in the ocular tissue.

In one embodiment, an effective amount of the nanoparticles are transported from the bloodstream into the brain tissue and accumulate in the brain tissue or wherein an effective amount of the nanoparticles are transported from the brain tissue into the bloodstream and accumulate in the bloodstream.

The formulations of R-targeted nanoparticles described herein can be used for the selective tissue delivery of a therapeutic, prophylactic, or diagnostic agent to an individual or patient in need thereof. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate enteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

The present invention will be further understood by reference to the following non-limiting examples.

### EXAMPLES

All animal studies were conducted under the supervision of MIT's Division of Comparative Medicine in compliance with the NIH's Principles of Laboratory Animal Care.

### Example 1

### FcRn Expression in Wild-Type Mice

Materials and Methods

For western blot analysis, sections of small intestine and colon were removed from wild-type mice after euthanasia. Intestinal epithelial cells were isolated and the protein was extracted (Booth, in Culture of Epithelial Cells, Wiley-Liss, Inc., 2002, pp. 303-335 and Pan et al., PloS One 7: e30247 (2012)). Protein concentrations in the extracts were determined using the BCA assay. Extracts were resolved on a 12% SDS-PAGE gel under reducing conditions. Proteins were transferred onto a nitrocellulose membrane. The membrane was blocked with 5% nonfat milk, probed with rabbit anti-mouse FcRn (Santa Cruz Biotech) for 1 h, and then incubated with goat anti-rabbit IgG-HRP (Santa Cruz Biotech). All blocking, incubations, and washes used PBS-T (PBS with 0.05% Tween 20). Detection was by chemiluminescence. Band intensity was quantified using ImageJ.

The immunohistochemistry was studied on small intestine tissues harvested and fixed in 10% formalin overnight. After ethanol dehydration, tissues were paraffin embedded and cut into 8 μm-thick sections, mounted on slides, and dried overnight. The tissues were then rehydrated using xylene and ethanol. Endogenous peroxidase activity, endogenous biotin, and nonspecific proteins were blocked with 3% H2O2, avidin blocking agent, and 10% goat serum, respectively. The samples were incubated with polyclonal rabbit IgG or anti-mouse FcRn IgG (Santa Cruz Biotech) primary antibody overnight, then incubated with biotinylated anti-rabbit secondary antibodies (Santa Cruz Biotech), with streptavidin-HRP, developed with DAP, and mounted with hematoxylin counterstain.

Results

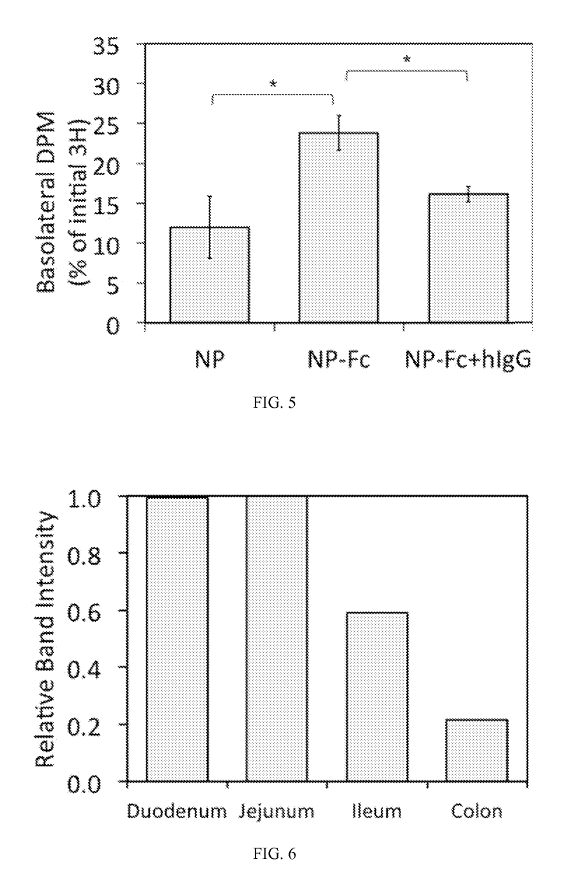

Western blot analysis confirmed FcRn expression throughout the entire small and large intestine of wild-type mice. Quantification of band intensity is shown in FIG. 6. The results demonstrate that the FcRn expression is highest in the upper sections of the small intestine (duodenum and jejunum) and decreased in the distal sections of the small intestine and the colon. Immunohistochemistry showed that FcRn was localized to the epithelium of the intestinal villi of the duodenum of wild-type mice (FIG. 3C). Immunohistochemistry of mFcRn imaged in sections of mouse duodenum appeared as brown against a negative control of tissue stained with polyclonal IgG.

### Example 2

### Preparation of Fc-Targeted Nanoparticles

Materials and Methods

NPs were formed from biodegradable and biocompatible amphiphilic poly(lactic acid)-b-poly(ethylene glycol) (PLA-PEG) block copolymers. PLA is a biodegradable polymer used in many FDA-approved products and forms the NP core owing to its hydrophobicity. PEG is a biocompatible polymer that remains on the NP surface owing to its hydrophilic nature and forms the NP corona. PLA-PEG was synthesized using ring-opening polymerization with a free terminal maleimide group (PLA-PEG-MAL) to conjugate the Fc portion of IgG. D,L-Lactide (Sigma-Aldrich) and MAL-PEG-OH (JenKem Technology) were used to synthesize PLA-PEG-MAL by ring opening polymerization. D,L-Lactide (3 g, 20.8 mmol) and MAL-PEG-OH (544 mg, 0.16 mmol) were dissolved in 15 mL anhydrous toluene in a round bottom flask. Tin(II) ethylhexanoate (38 mg, 0.09 mmol) was then added. The flask with condenser was placed in an oil bath, purged with nitrogen for 10 minutes, heated to 120° C., and reacted overnight while 4° C. water circulated through the condenser. Toluene was then evaporated and the polymer precipitated in a 50:50 (v/v) mixture of ice-cold methanol and diethyl ether and vacuum-dried. The PLA-PEG-MAL was characterized by ¹H NMR (400 MHz), δ=5.28-5.11 (br, —OC—CH(CH₃)O— in PLA), 3.62 (s, —CH₂CH₂O— in PEG), 1.57-1.45 (br, —OC—CHCH₃O— in PLA). Using GPC, the polymer Mₙ=12.5 kDa with M_{w}/Mₙ=1.47 relative to polystyrene standards.

The nanoprecipitation self-assembly method was used to generate PLA-PEG-MAL nanoparticles (Bilati, et al., AAPS PharmSciTech, 6:E594-E604 (2005)). 3 mg PLA-PEG-MAL was dissolved in 300 μL acetonitrile and added dropwise to 1.5 mL water. The solution was mixed for 2 h, and the NPs were purified by filtration using Millipore Amicon Ultra 100,000 NMWL. The NPs were washed 2× with water and 2× with phosphate-buffered saline (PBS) containing 5 mM EDTA. Particle diameter and surface charge (zeta potential) were measured using dynamic light scattering with a Brookhaven Instruments ZetaPALS (See FIG. 3).

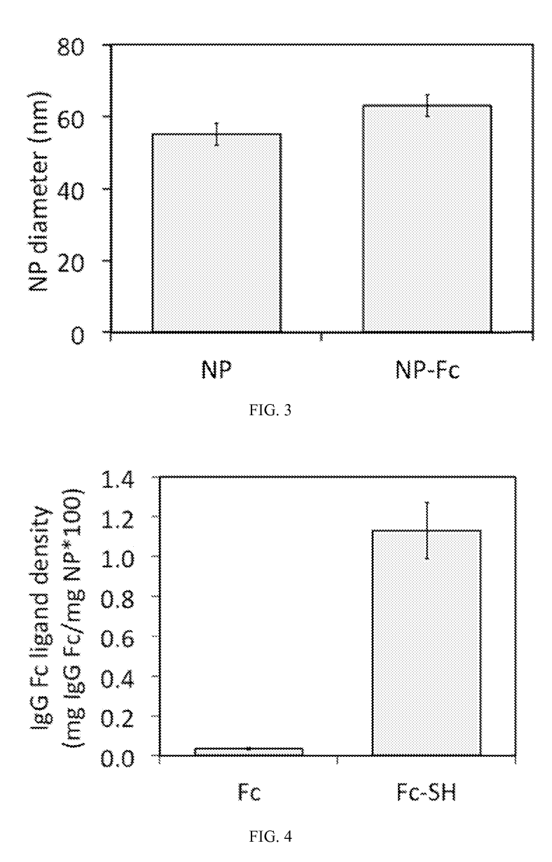

To prepare the Fc-targeted nanoparticles, polyclonal IgG Fc fragments were covalently conjugated to PEG using maleimide-thiol chemistry. 2-Iminothiolane was used to modify the Fc with thiol groups (Fc-SH). 86 μg of purified human polyclonal IgG Fc prepared by papain digestion (Bethyl Laboratories) or 95 μg of chicken IgY Fc (Jackson ImmunoResearch Laboratories) in PBS containing 5 mM EDTA was reacted with 0.48 μL of 5 mg/mL 2-iminothiolane (Traut's Reagent) for 1 h. Fc-SH was incubated with PLA-PEG-MAL NPs for conjugation. The modified Fc was added to the NPs and mixed for 1 h to allow conjugation at 4° C. The Fc-targeted nanoparticles were washed with PBS using Millipore Amicon Ultra 100,000 NMWL. The conjugation of IgG Fc to the NP surface was measured using a protein bicinchoninic acid (BCA) assay from Lamda Biotech. Particle diameter and surface charge (zeta potential) were measured using dynamic light scattering with a Brookhaven Instruments ZetaPALS. The amount of Fc conjugated to the NPs was measured for both Fc-SH and unmodified Fc (See FIG. 4).

Results

The PLA-PEG-MAL nanoparticles had a mean hydrodynamic diameter of 55 nm and a polydispersity of 0.05 The hydrodynamic diameter of the Fc-targeted nanoparticles increased from 55 nm (PLA-PEG-MAL particles) to 63 nm after Fc conjugation (FIG. 3)—an increase consistent with the hydrodynamic diameter of IgG Fc (˜3 nm) (Armstrong, et al., Biophys. J. 87:4259-4270 (2004)). Unmodified Fc resulted in a lower ligand density than Fc-SH, indicating minimal nonspecific interactions between Fc and the NP surface and that the unbound Fc was successfully separated from NP using centrifugal filtration. The ligand density for Fc-SH was 32-fold higher than unmodified Fc, indicating that Fc was bound on the nanoparticle surface. The surface charge showed only a minor change from −4.3±0.4 for NP to −5.6±1.1 mV for NP-Fc (mean±SD, n=3, p>0.05, Student's t test).

### Example 3

### In Vitro Transepithelial Transport of Fc-Targeted Nanoparticles

Materials and Methods

In vitro NP transepithelial transport studies were conducted using an epithelial cell monolayer model with Caco-2 cells, a human epithelial colorectal adenocarcinoma cell line typically used as a model of the intestine for drug permeability testing. Caco-2 cells endogenously express human FcRn and human beta-2-microglobulin, and have previously been used for IgG transcytosis studies (Dickinson, et al., J. Clin. Invest., 104:903-911 (1999) and Liu et al., J. Immunol., 179:2999-3011 (2007)). Transepithelial transport studies utilized Transwell plates (Costar) with a Caco-2 (American Type Culture Collection—ATCC) cell density of 5.5×10⁴ in media [ATCC formulated Eagle's Minimum Essential Medium with aqueous penicillin G (100 units/mL), streptomycin (100 U/mL), and fetal bovine serum (FBS, 20%)]. On the day of the transport experiment, the media was changed to HBSS pH 6.5 in the apical chamber and HBSS pH 7.4 in the basolateral chamber and allowed to equilibrate for 1 h at 37° C. and 5% CO₂. Prior to the experiment and at the end of the experiment, the monolayer integrity was checked by measuring the transepithelial resistance (TEER) using a Millicell-ERS (Millipore). TEER values were 900-1000Ω/cm² for wells used in transport experiments and the TEER remained constant throughout the experiments. ³H-labeled NPs were prepared by blending 50 μg ³H-poly(lactic-co-glycolic acid) (PLGA) (Perkin Elmer) with 1 mg PLA-PEG-MAL in 100 μL acetonitrile prior to nanoprecipitation in 500 μL water and then washed in HBSS pH 6.5 prior to the transport experiment. The apical solution was then replaced with a solution of 100 μg ³H-labeled NPs or NP-Fc in 250 μL HBSS pH 6.5. The NP formulations were incubated for 24 h before measuring the ³H content in the basolateral chamber. The basolateral solution was collected and added to a Hionic-Fluor scintillation cocktail (Perkin Elmer) before analysis using a Packard Tri-Carb Scintillation Analyser. At the end of the experiment, the TEER was measured again to verify monolayer integrity. For the IgG Fc blocking experiment, a 50× molar excess of IgG Fc relative to Fc on the NP-Fc surface was added concurrently with NP-Fc to the apical chamber, and the ³H content in the basolateral chamber was measured after 24 h.

Results

³H-labeled NPs were successfully used to measure transport across the Caco-2 monolayer. The pH gradient established from the apical to basolateral side of the Caco-2 polarized monolayer mimicked the physiological pH of the duodenum and enhanced apical binding. The transcytosis of non-targeted nanoparticles (control) and Fc-targeted nanoparticles was measured. ³H measurements for Fc-targeted nanoparticles on the basolateral side were twofold greater than non-targeted nanoparticles after 24 h, indicating that Fc on the nanoparticle surface significantly enhanced transepithelial transport in vitro (See FIG. 5). When Fc-targeted nanoparticles were combined with a 50-fold excess of free IgG Fc as a blocking agent for the FcRn transcytosis pathway, transport was significantly reduced, indicating that the enhanced transport of Fc-targeted nanoparticles is at least partially receptor-mediated (FIG. 5).

### Example 4

### In Vivo Absorption and Biodistribution of Fc-Targeted Nanoparticles in Wild-Type Mice

Materials and Methods

The biodistribution and absorption efficiency of both targeted and non-targeted NPs were quantitatively measured by radiolabeling the NPs with ¹⁴C. To prepare ¹⁴C-labeled NPs, 450 μg PLA-¹⁴C was blended with 3 mg PLA-PEG-MAL in 300 μL acetonitrile prior to nanoprecipitation in 1.5 mL water. NPs were washed 2× with water and 2× with PBS. ¹⁴C release was measured by preparing a batch of ¹⁴C NPs in PBS with pH 7.4 and dividing the batch equally into 500 μg samples for incubation at 37° C. At each timepoint, samples were collected, washed 3× with PBS using Millipore Amicon Ultra 100,000 NMWL, and then added to 15 mL of Hionic Fluor scintillation cocktail. The activity was counted using a Packard Tri-Carb Scintillation Analyser. For the in vivo biodistribution experiments, 6-12 week old wild-type mice were fasted 8 h prior to oral gavage of 1.5 mg (0.1 μCi/mouse) of 14C-labeled NP and NP-Fc in 7 mL PBS/kg. Groups of mice (n=5 mice) were euthanized at each time point, and the spleen, kidneys, liver, lungs, and heart were harvested. Each organ was placed directly in a scintillation vial except for the liver, which was homogenized and ˜100 mg was analyzed. Each organ was solubilized in 2 mL of Solvable (Perkin Elmer) for 12 h at 60° C. and then decolored with 200 μL of 0.5 M EDTA (Invitrogen) and 200 μL 30% hydrogen peroxide (Fisher Scientific) for 1 h at 60° C. The activity was counted in 15 mL Hionic-Fluor scintillation cocktail using a Packard Tri-Carb Scintillation Analyser. To determine 100% dose, vials of 500 μg NPs and NP-Fc were counted in 15 mL Hionic-Fluor scintillation cocktail. For the oral absorption efficiency, total ¹⁴C counted in all tissues was added at each time point. The AUC of total absorbed ¹⁴C versus time was calculated using the trapezoid method and divided by the initial dose to determine the oral absorption efficiency. The results were reported as mean±SEM, and comparison of non-targeted NPs and NP-Fc utilized the two-tailed Student's t-test.

In vivo transport of NP-Fc across the intestinal epithelium was visualized using fluorescently labeled NPs. Fluorescently-labeled NPs were prepared by blending 100 μg PLA-AF647 with 1 mg PLA-PEG-MAL in 100 μL acetonitrile prior to nanoprecipitation in 500 μL water. Fluorescently-labeled NP and NP-Fc were washed 3× in water until the flow-through was clear and suspended in 200 μL water (7 mL water/kg). The suspension was then administered to the mice by oral gavage. Wild-type Balb/c mice (Charles River Laboratories) (n=3) were fasted overnight prior to gavage. After 1.5 h, the mice were euthanized. Duodenum tissue sections were frozen into Tissue-Tek OCT using liquid nitrogen. Cross sections of the tissue were obtained using a Leica CM1900 Cryostat with a thickness of 12 μm. The tissue was air-dried overnight and then stained with Prolong Gold (Life Technologies) antifade reagent with 4′,6-diamidino-2-phenylindole (DAPI). Fluorescent images were obtained using a Zeiss LSM 710 NLO scanning confocal microscope under oil immersion at 40× magnification.

Results

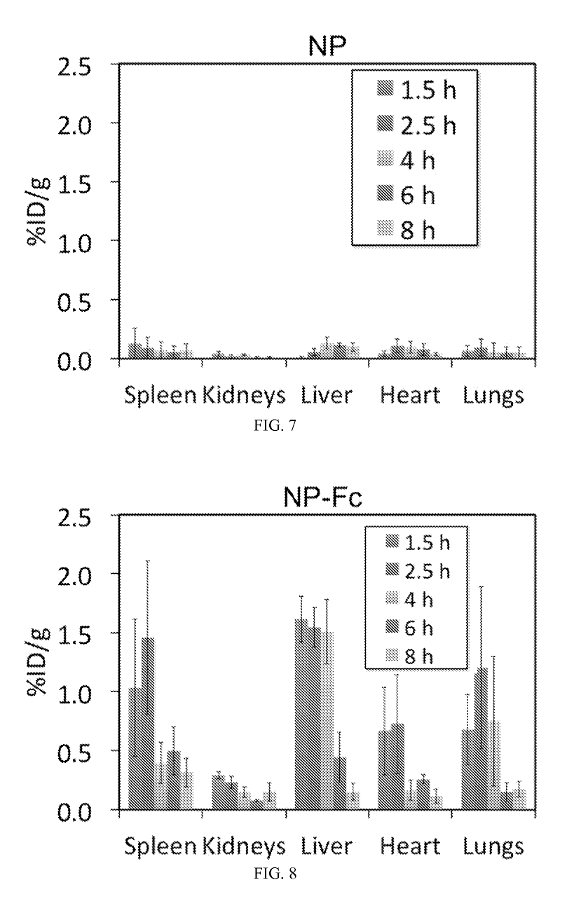

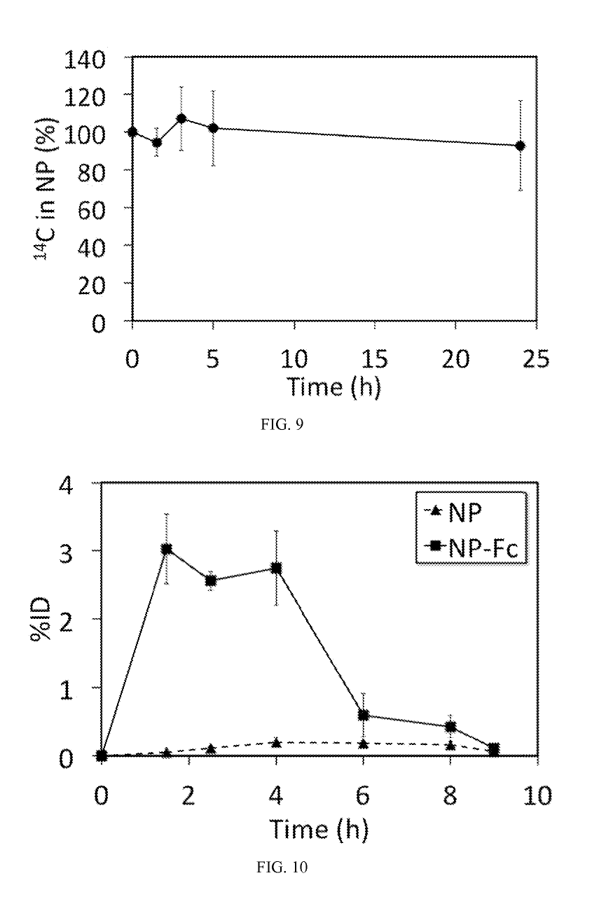

To ensure that the ¹⁴C remained with the NPs over the course of the experiment, the release of ¹⁴C from the NPs was measured and no release was observed over 24 h (See FIG. 9). FIG. 7 shows the biodistribution of non-targeted nanoparticles and FIG. 8 shows the biodistribution of Fc-targeted nanoparticles NP-Fc over the course of 8 hours after oral administration to fasted wild-type mice. For the non-targeted NPs, a small amount of ¹⁴C was measured in the organs. By contrast, a large amount of ¹⁴C was measured in the spleen, kidneys, liver, and lungs for Fc-targeted nanoparticles, indicating that NP-Fc entered systemic circulation after oral administration and reached several organs known to express FcRn. The ¹⁴C in the organs was transient, peaking at 2.5 h post-delivery and clearing from the organs at later time points (FIG. 8). The total ¹⁴C absorbed over time was calculated by summing the ¹⁴C measured in each of the organs (spleen, kidneys, liver, heart, and lungs) at a specific time point (FIG. 10). Significantly higher amounts of ¹⁴C were absorbed for Fc-targeted nanoparticles at 1.5, 2.5, and 4 h compared with non-targeted nanoparticles, indicating that Fc targeting enhanced absorption. The area under the curve (AUC) was used to calculate the oral absorption efficiency, which was 1.2%*h±0.2 for non-targeted NPs and 13.7%*h±1.3 for Fc-targeted nanoparticles (mean±SEM with n=5 mice per timepoint; P<0.001, Student's t test). This difference in AUC suggests that IgG Fc targeting was responsible for an 11.5-fold increase in NP absorption.

Fasted wild-type mice were orally administered the fluorescently labeled nanoparticles, and sections of the duodenum were collected and analyzed using confocal fluorescence microscopy. Confocal fluorescence images of 12-m sections of mouse duodenum Cell nuclei were stained with DAPI. The images were compared for both non-targeted NPs and NP-Fc. For the non-targeted NPs, the fluorescence from the NPs was not observed in the villi, suggesting that the NPs were unable to enter the villi. However, for the Fc-targeted nanoparticles, fluorescence was observed inside the villi on the basolateral side of the epithelial cells, indicating that Fc-targeted nanoparticles crossed the intestinal epithelium and entered the lamina propria.

### Example 5

### Oral Insulin Delivery to Wild-Type Mice with Fc-Targeted Nanoparticles

Materials and Methods

Insulin NPs were prepared by blending 900 μg human recombinant insulin (Sigma-Aldrich), 450 μg PLGA (50:50 G:L, inherent viscosity 0.20 dL/g) with terminal carboxylate groups (Lactel), and 3 mg PLA-PEG-MAL together in 525 μL dimethyl sulfoxide (DMSO) prior to nanoprecipitation in 2.1 mL water. Free insulin was removed using Millipore Amicon Ultra 100,000 NMWL by washing 2× with water and 2× with PBS. Insulin encapsulation was measured by heating the NPs to 60° C. for 30 min, and insulin was quantified using a BCA assay or insulin ELISA kit (Millipore). Insulin release from the NP was measured by dividing a batch of insNP equally into 24 kDa dialysis units (Pierce) and incubating at 37° C. in PBS with pH 7.4. At each timepoint, three samples of insNP were collected, washed with PBS using Millipore Amicon Ultra 100,000 NMWL, heated to 60° C. for 30 min, and measured for insulin using a BCA assay.

To determine the bioactivity, insulin released from insNP was collected and injected into fasted wild-type mice by tail-vein injection. The bioactivity was measured by monitoring the blood glucose and comparing the response to an equivalent dose of free insulin solution (3.3 U/kg). Wild-type mice were fasted for 8 h, and the mice (n=3/group) used were chosen so that the mean initial blood glucose levels were the same for each group. 3.3 U/kg of released insulin was administered to the fasted mice by tail-vein injection. An equivalent dose of free insulin by mass was administered by tail-vein injection to another group of fasted mice. The blood glucose level was measured using the Contour blood glucose monitor (Bayer).

The hypoglycemic response generated after oral administration of the targeted insNP (insNP-Fc) was tested using fasted wild-type mice and compared with the efficacies of non-targeted insNP, free insulin, and NP-Fc without insulin. 6-12-week old wild-type or FcRn knockout mice (Jackson Laboratories) were fasted for 8 hours. Mice (n=5-6) were chosen per group such that the mean initial blood glucose levels were the same per group. 150 or 250 μg of insNP or insNP-Fc (insulin dose—0.66 or 1.1 U/kg) in 7 mL PBS/kg were administered to the mice by oral gavage. For controls, 1.1 U/kg of free insulin and 250 μg of NP-Fc without insulin in 7 mL PBS/kg were administered by oral gavage. For the excess IgG Fc control, 250 μg of insNP-Fc was formulated with 50× molar excess of IgG Fc in 7 mL PBS/kg prior to administration to the mice by oral gavage The blood glucose level was measured as described above.

Results

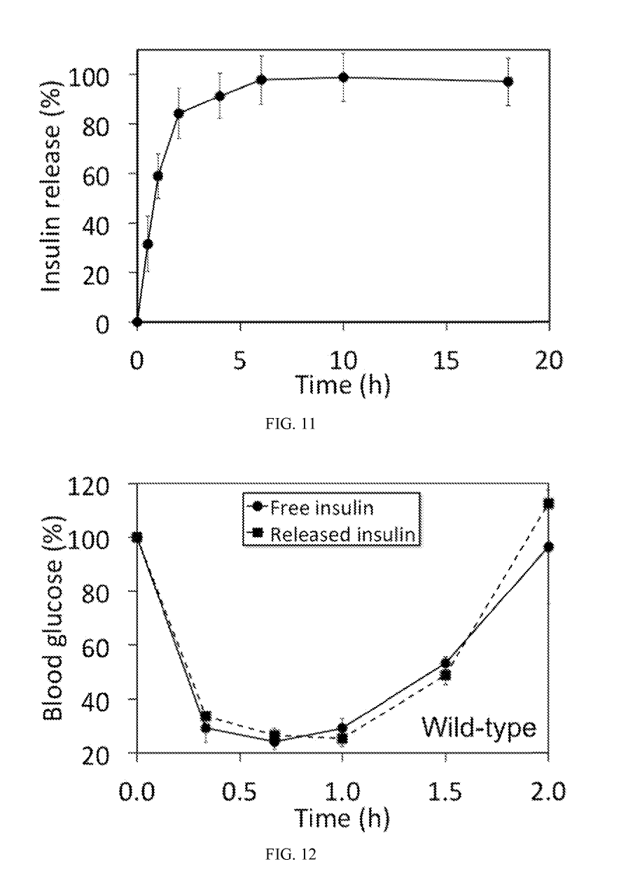

Fc-targeted nanoparticles that are capable of encapsulating insulin were developed as a model NP-based therapeutic for diabetes that could be orally administered and evaluated for eliciting a pharmacologic response. Insulin NPs prepared as described above resulted in an insulin load of 0.5% (w/w). The particle size remained small (mean hydrodynamic diameter, 57 nm) while still allowing insulin encapsulation. The release of insulin from insNP in PBS at 37° C. demonstrated a strong burst release in the first hour followed by a controlled release (See FIG. 11). The insulin release profile was advantageous because it allowed all of the insulin to be delivered before complete clearance of the particles after about 10 h.

The bioactivity of insulin released from insNP was compared to an equivalent dose of free insulin solution (3.3 U/kg). The released insulin generated a similar hypoglycemic response in mice (See FIG. 12), indicating that the encapsulated insulin was bioactive after release from the insNP.

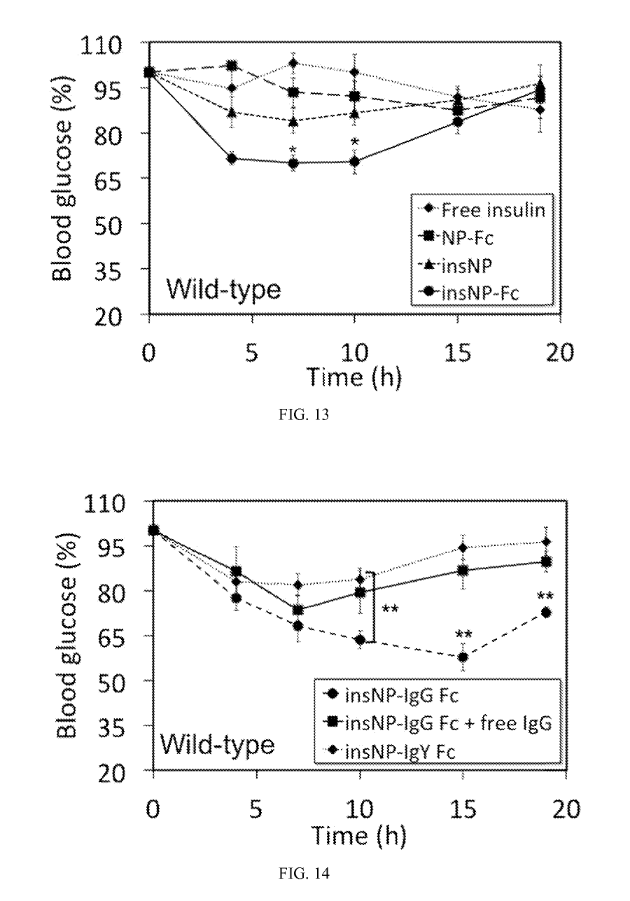

FIG. 13 compares the blood glucose response in wild-type mice upon oral administration of various formulations. The free insulin administered orally did not generate a glucose response in the mice, unlike the free insulin injected into the tail vein that was able to generate a glucose response (FIG. 12). The Fc-targeted nanoparticles without insulin also were unable to generate a glucose response. The glucose response generated by non-targeted insNP was not different from that generated by the control groups at any time point. However, Fc-targeted nanoparticles containing insulin caused a significant hypoglycemic response in the mice, reducing the glucose during the first 10 h after administration. This is consistent with the biodistribution (FIG. 8) and insulin release data (FIG. 11), which demonstrated that the particles were cleared and the insulin was released within 10 h, respectively. The blood glucose level then increased and was similar to that of the control groups by 15 h. The insNP-Fc insulin dose required to generate the hypoglycemic response was 1.1 U/kg, which is clinically relevant (See Cochran et al., Diabetes Care 28:1240-1244 (2005)) and lower than other oral insulin delivery systems that require 10-100 U/kg to generate a glucose response (See Chen, et al., Biomaterials 32:9826-9838 (2011)). When compared with the glucose response from free insulin administered by tail vein injection (FIG. 12), the orally administered insNP-Fc resulted in a prolonged (15 h vs. 1.5 h) hypoglycemic response (FIG. 13).

To demonstrate that the enhanced hypoglycemic response generated by insNP-Fc was due specifically to the IgG Fc ligand on the NP surface, several additional control groups were tested. The first control was to administer insNP-Fc concurrently with a 50-fold excess of free IgG Fc. The second control was to conjugate chicken IgY Fc fragments to insNP instead of human IgG Fc fragments. [Chicken IgY is the functional equivalent of IgG in non-mammalian species such as birds, but does not bind to mouse FcRn (See Israel, et al., Immunology 89, 573-578 (1996)).] Both control groups had hypoglycemic responses that were significantly less than insNP-Fc (See FIG. 14), indicating that the use of the IgG Fc as a targeting ligand was responsible for the enhanced hypoglycemic response.

### Example 6

### Oral Insulin Delivery to FcRn Knockout Mice

Materials and Methods

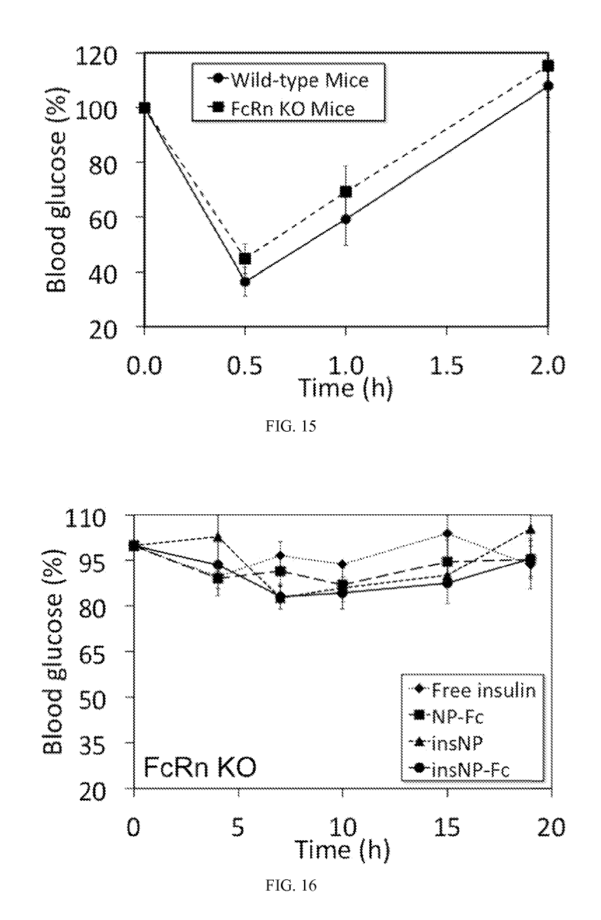

The role of FcRn in NP transepithelial transport was tested by repeating the efficacy experiment using FcRn knockout (KO) mice. FcRn KO mice had the same insulin sensitivity as the wild-type mice, so the same insulin dose was used for both strains (FIG. 15).

Results

In contrast to the results in the wild-type mice (FIG. 13), insNP-Fc did not generate a hypoglycemic response significantly different from the other three groups in the FcRn KO mice (FIG. 6). In these FcRn KO mice, the response generated by insNP-Fc resembled the response generated by non-targeted insNP in the wild-type mice (FIG. 13), suggesting that the benefit gained from using Fc was specifically due to FcRn.

### Example 7

### In Vivo Glucose Response Dose-Dependency

Materials and Methods

The glucose response in both wild-type and FcRn KO mice was evaluated for dose dependency using two different doses of insNP-Fc: 0.66 U/kg and 1.1 U/kg.

Results

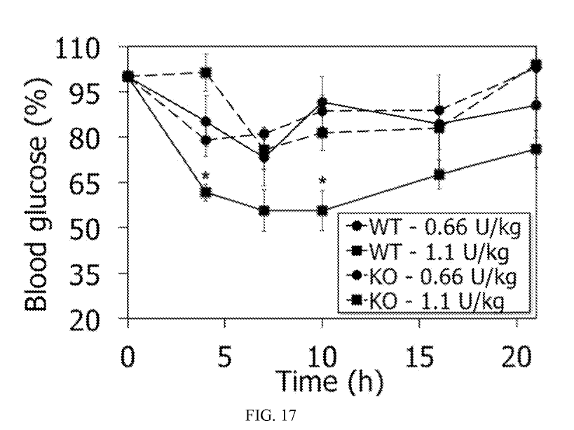

See FIG. 17. For the FcRn KO mice, there was no difference in the glucose response between the two doses of insNP-Fc. However, for the wild-type mice, the glucose response was significantly greater at the higher dose of insNP-Fc, suggesting a possible dose-dependence in the wild-type mice.

1. A nanoparticle formulation for transport of agents through tissue, tissue barriers, and tissue linings comprising an effective amount of polymeric nanoparticles comprising
an outer surface comprising a blend of a first amphiphilic block co-polymer comprising a hydrophobic block and a hydrophilic block with a targeting moiety conjugated thereto, and a second polymer selected from an amphiphilic block co-polymer and a hydrophobic polymer, wherein the targeting moiety is absent from the second polymer; and
a core comprising a therapeutic, prophylactic, or diagnostic agent,
wherein the targeting moiety is present on the exterior surface of the nanoparticles and can bind to a receptor on the surface of the cells in the tissue to effect transcytosis of the nanoparticles into and through the cells.

2. The nanoparticle formulation of claim 1 for delivery into and through heart, skeletal muscle, or adipose tissue, wherein the receptors are selected from the group consisting of gp60 and ligands for FcRn.

3. The nanoparticle formulation of claim 1 for delivery into and through testis tissue, wherein the receptors are selected from the group consisting of chorionic gonadotropin receptor, Insulin receptor and insulin-like growth factor receptor, FcRn, and Transferrin receptor.

4. The nanoparticle formulation of claim 1 for delivery into and through brain tissue, wherein the receptors are selected from the group consisting of insulin receptor; insulin-like growth factor receptor; LDL receptor-related proteins 1 and 2 (LRP1 and LRP2); LDL receptor; Diptheria toxin receptor; Transferrin; Receptor for advanced glycation end products (RAGE); Scavenger receptor (SR); and ligands for FcRn.

5. The nanoparticle formulation of claim 1 for delivery into and through intestinal tissue, wherein the receptors are selected from the group consisting of receptors for M cells; Terminal galactose (ricin B receptor); aminopeptidase N; pIgA receptor; FcRn; CD23 (for IgE); and Cubulin/Megalin (vitamin B12).

6. The nanoparticle formulation of claim 1 for delivery into and through liver tissue, wherein the receptor is pIgA or FcRn.

7. The nanoparticle formulation of claim 1 for delivery into and through kidney tissue, wherein the receptors are selected from the group consisting of pIgA, Megalin, FcRn, and Terminal galactose (ricin B receptor).

8. The nanoparticle formulation of claim 1 for delivery into and through placental tissue, wherein the receptors are selected from the group consisting of aminopeptidase N, pIgA, FcRn, Transferrin, and Megalin.

9. The nanoparticle formulation of claim 1 for delivery into and through lung tissue, wherein the receptors are selected from the group consisting of ligands for FcRn; Transferrin; Terminal galactose (ricin B receptor); pIgA; FcRn; CD23 (for IgE); and gp60.

10. The nanoparticle formulation of claim 1 for delivery into and through mammary gland tissue, wherein the receptors are selected from the group consisting of gp60; aminopeptidase N; pIgA; FcRn; and Transferrin.

11. The nanoparticle formulation of claim 1 for delivery into and through thyroid tissue, wherein the receptor is gp60 and Megalin.

12. The nanoparticle formulation of claim 1 for delivery into and through genitourinary tract tissue, wherein the receptors are selected from the group consisting of pIgA, Transferrin, Megalin; gp340; FcRn; and lutropin receptor.

13. The nanoparticle formulation of claim 1 wherein the receptors are ligands for FcRn.

14. The nanoparticle formulation of claim 1, wherein the targeting moieties are selected from the group consisting of proteins, peptides, amino acids, lipid, carbohydrate, nucleic acid, small molecules, and combinations thereof.

15. The nanoparticle formulation of claim 13, wherein the targeting moieties are antibodies or fragments thereof binding to FcRn.

16. The nanoparticle formulation of claim 13, wherein the FcRn receptor targeting moieties are IgG (all isotypes) Fc fragments engineered to have altered binding to the FcRn or reduce immunogenicity.

17. The nanoparticle formulation of claim 16, wherein the IgG Fc has mutations in the CH2 and CH3 domains.

18. The nanoparticle formulation of claim 13, wherein the FcRn targeting moieties are IgG (all isotypes) Fc fragments engineered with distinct mutations, deletions or additions of amino acids, and are 95%, 90% or 85% homologous to the Fc fragment.

19. The nanoparticle formulation of claim 16, wherein IgG (all isotypes) Fc fragments have one or more of the mutations in the CH2 and CH3 domain selected from the group consisting of T250Q/M428L, M252Y/S254T/T256E+H433K/N434F, E233P/L234V/L235A/?G236+A327G/A330S/P331S, K322A, and L235E+E318A/K320A/K322A.

20. The nanoparticle formulation of claim 1 comprising two or more types of receptor binding moieties.

21. The nanoparticle formulation of claim 1, wherein the nanoparticle comprises one or more targeting moieties targeting a specific organ, tissue, cell type, or subcellular compartment.

22. The nanoparticle formulation of claim 1 comprising a targeting moiety binding to a target that does not mediate transcytosis into the cell.

23. (canceled)

24. The nanoparticle formulation of claim 1, wherein the targeting moieties are present in a density greater than about 1 mg targeting moiety to about 500 mg particle or at least 10 moieties per square micron.

25. The nanoparticle formulation of claim 1, wherein the targeting moieties are present on the surface of the nanoparticles in a density greater than about 1,000 moieties per square micron.

26-27. (canceled)

28. The nanoparticle formulation of claim 1 wherein a targeting moiety that effects transcytosis of the nanoparticles is released from the nanoparticle surface after the nanoparticle crosses the tissue, tissue barrier, or tissue lining, optionally by a change in pH, change in temperature, enzymatic degradation, change in flow shear rate, change in magnetic field, change in electric field, or change in ionic strength.

29. The nanoparticle formulation of claim 1, wherein the therapeutic, prophylactic or diagnostic agent is released by a change in pH, change in temperature, enzymatic degradation, change in flow shear rate, change in magnetic field, change in electric field, or change in ionic strength.

30-32. (canceled)

33. The nanoparticle formulation of claim 1, wherein the hydrophobic block comprises a polymer selected from the group consisting of polyhydroxyacids, polyhydroxyalkanoates, polycaprolactones, poly(orthoesters), polyanhydrides; poly(phosphazenes), poly(lactide-co-caprolactones), polycarbonates, polyesteramides, polyesters, poly(dioxanones), poly(alkylene alkylates), polyethers, polyurethanes, polyetheresters, polyacetals, polycyanoacrylates, polyacrylates, polymethylmethacrylates, polysiloxanes, polyketals, polyphosphates, polyhydroxyvalerates, polyalkylene oxalates, polyalkylene succinates, poly(maleic acids), and copolymers thereof.

34. The nanoparticle formulation of claim 33, wherein the hydrophobic block comprises a polymer selected from the group consisting of poly(lactic acid), poly(glycolic acid), and poly(lactic acid-co-glycolic acids).

35. The nanoparticle formulation of claim 1, wherein the hydrophilic block comprises a polymer selected from the group consisting of cellulosic polymers, polypeptides, poly(amino acids), polyalkylene glycols, polyalkylene oxides, poly(hydroxy acids); poly(vinyl alcohols), and copolymers thereof.

36. (canceled)

37. The nanoparticle formulation of claim 1 comprising a lipid disposed between the outer surface and the core of the nanoparticles.

38. The nanoparticle formulation of claim 1, wherein the targeting moieties are adsorbed, absorbed, conjugated, complexed, bound, or assembled into the nanoparticle or a component thereof prior to or after formation of the nanoparticles.

39. The nanoparticle formulation of claim 1 comprising a polyalkylene oxide surface on the nanoparticles.

40. The nanoparticle formulation of claim 1 wherein the nanoparticles have a diameter of between 3 and 500 nm, preferably between 10 and 150 nm.

41-82. (canceled)
